(19) United States
(12) Patent Application Publication
(10) Pub. No.: US 2009/0280182 A1
(43) Pub. Date: Nov. 12, 2009

(54) Devices for the treatment of wounds and methods and kits therefor

(76) Inventors: Stayce Beck, Menlo Park, CA (US); David Myung, Santa Clara, CA (US); Curtis W. Frank, Cupertino, CA (US); Jennifer R. Cochran, Stanford, CA (US); Michael T. Longaker, Atherton, CA (US); George P. Yang, San Francisco, CA (US); Daphne P. Ly, Palo Alto, CA (US); Shira G. Mandel, Palo Alto, CA (US)

Correspondence Address: LUMEN PATENT FIRM, 350 Cambridge, Suite 100, PALO ALTO, CA, 94306, US

(21) Appl. No.: 12/387,318

(22) Filed: Apr. 29, 2009

Related U.S. Application Data
(60) Provisional application No. 61/126,103, filed on Apr. 30, 2008.

(51) Int. Cl. A61K 9/10; A61K 38/16

(52) U.S. Cl. 424/486; 514/12

(57) ABSTRACT

Interpenetrating network hydrogels are described that may be incorporated into wound dressings and/or in implants. The properties of the interpenetrating network hydrogel may be tuned to control an amount of moisture in a wound environment. The devices, methods, and kits described herein may be adapted to treat a variety of wound types at a variety of healing stages over a range of time scales. Some hydrogels may be configured to deliver one or more vulnerary agents to a wound. The interpenetrating network hydrogels may also be adapted to control a rate and/or amount of moisture uptake so that the hydrogels may be used as expandable implants to expand tissue.

### CROSS-REFERENCE TO RELATED APPLICATIONS

This application claims priority from U.S. Provisional Application Nos. 61/126,103, filed on Apr. 30, 2008, which is incorporated herein by reference.

### FIELD OF THE INVENTION

The devices, kits and methods described herein are in the field of wound healing, wound treatment, and wound management. In some variations, the devices, kits and methods relate to wound debridement, to tissue expansion, and/or to the delivery of a vulnerary agent to a wound.

### BACKGROUND OF THE INVENTION

Many wounds and ulcers can become chronic. That is, they may not heal over a long period of time (e.g., several months or years). Patients suffering from diabetes mellitus may be particularly susceptible to developing chronic wounds or ulcers. Elderly patients in general are more likely to develop chronic wounds. In addition, pressure ulcers can often become chronic, e.g., pressure ulcers developed by sedentary or bedridden patients.

Normal wound healing follows a sequence of stages, generally described as follows. First, the inflammatory stage begins immediately after a surgical or traumatic injury and generally lasts a few days. During the inflammatory stage, the body operates to remove dead or damaged tissue and contaminants from the wound site. The proliferative stage follows the inflammatory stage. During the proliferative stage, which generally lasts a week, but may last several weeks, cells (e.g., fibroblasts) proliferate and migrate, and collagen and proteoglycan are produced. The extracellular matrix is also synthesized during the proliferative stage, which provides a structural framework to support cells and tissue during wound healing. Finally, wound healing enters the remodeling stage, during which the extracellular matrix is reorganized and cross-linked to increase mechanical strength. The remodeling stage can take place over months, e.g., up to a year, or even longer.

Thus, during normal healing of a wound, the wound tissue undergoes both degradative and constructive processes. For chronic wounds, degradative processes can overwhelm the constructive processes such that the wound persists. Infection also frequently develops in persistent wounds, further impeding the healing process. If infection cannot be adequately controlled, tissue excision or amputation may be necessary.

The local environment of any wound, acute or chronic, during the inflammatory stage is hostile, being hypoxic and acidic and containing excess activated proteases to degrade damaged tissue. In chronic non-healing wounds, the hostile environment can persist unless treated. As such, the standard treatment to date for management of non-healing or chronic wounds has required medical practitioners to debride the wound of devitalized, contaminated or dead tissue and apply fresh saline-soaked dressing to the wound several times a day.

Vacuum-assisted closure (VAC) is a U.S. Food and Drug Administration approved treatment that involves applying a negative pressure environment to a wound to assist in draining the wound. VAC devices generally include a foam sponge positioned at the wound. An airtight seal is created over the sponge using adhesive, and the enclosed area including the sponge is connected to a vacuum source. Although some evidence of clinical benefit of VAC devices has been produced, these devices generally have limited applicability as they require the patient to be attached to a vacuum source.

In addition to debridement, wound healing agents can be used to treat or manage wounds. For example, growth factors can be applied to wounds. Epidermal growth factor (EGF), one of the major proteins involved in the re-epithelization and remodeling phase of wound healing, has been used in wound healing, e.g., to accelerate healing in gastric and oral ulcers, diabetic foot ulcers, skin graft donor sites, corneal epithelial wounds, and to treat perforations of tympanic membranes. Although clinical use for EGF has been demonstrated, improvements in practical application are needed. For example, EGF has a circulation half-life of about 3-7 minutes, which can limit its efficacy in wound healing. Recombinant platelet-derived growth factor (PDGF) has also been marketed for wound healing applications. However, recombinant PDGF remains expensive and the benefits of using recombinant PDGF in chronic wounds appear to be marginal over standard treatments involving wound debridement and dressing changes. The protease-rich wound environment and rapid in vivo clearance of small peptide molecules has led to the need for sustained-release or repetitive dosing schemes to achieve therapeutic benefit of EGF and other growth factors.

Therefore, a need exists for improved devices and methods to assist in wound healing and wound treatment or management. In particular, devices and methods adapted to treat chronic or non-healing wounds that are in need of debridement are needed.

### SUMMARY OF THE INVENTION

Described herein are devices, methods and kits for assisting in wound healing. The devices may comprise an interpenetrating network hydrogel capable of absorbing wound exudates. The properties of the interpenetrating network hydrogel utilized in the devices and related methods and kits may be tuned to control the amount of moisture in the wound environment as a function of time, e.g., by controlling a rate of moisture uptake and/or an amount of moisture uptake. Thus, the devices, methods and kits may be adapted to treat a variety of wound types at a variety of healing stages over a range of timescales. In some cases, the hydrogels may be configured to deliver one or more vulnerary agents to the wound in conjunction with controlling the moisture content of the wound. The devices, methods and kits described herein may be particularly useful for treating or managing chronic wounds.

Also described herein are implants for expanding tissue, and related methods and kits. The expanded tissue can then be used as graft tissue for another body region, or a prosthesis or other implant may be placed in the volume created by the tissue expansion. In general, the implants may comprise an interpenetrating network hydrogel. The hydrogel may be configured to absorb moisture from the body and/or from an external source to expand over time so as to expand tissue overlying the implant. The hydrogel may be selected to control the expansion of the tissue, e.g., by controlling a rate of moisture uptake and/or an ultimate size and/or shape of the implant. The implants may be designed to be temporary or long term implants. The physical properties of the hydrogel used in the implants may be tuned to have desired properties as a function of moisture content, e.g., Young's modulus, flexural modulus, shear modulus, tensile strength, compression strength, stiffness, and the like.

In general, the devices for assisting in wound healing comprise an interpenetrating polymer network (IPN) hydrogel. The IPN hydrogel comprises a first polymer network comprising a nonionic hydrophilic polymer covalently cross-linked between ends and a second polymer network that has been cross-linked and/or polymerized in the presence of the first polymer network. The hydrogel is configured to absorb wound exudates from a wound, and is pre-wetted with a predetermined amount of a wetting agent so as to be nonadhesive to surrounding tissue. For example, some variations of devices may comprise at least about 10% by weight of the wetting agent. The wetting agent may be any suitable substance, e.g., a saline solution or glycerol. In some variations, the devices may be configured to be removably secured to skin proximate the wound.

In some devices, the IPN hydrogel may comprise a first polymer network that comprises poly(ethylene glycol) (PEG) or derivatives there of. In these devices, the molecular weight of a segment of the PEG between cross-links may be about 1 kDa to about 20 kDa, or from about 3 kDa to about 8 kDa. In some variations, the IPN hydrogel may comprise a second polymer network that is hydrophilic, and in some cases, ionizable, e.g., poly(acrylic acid) (PAA).

In certain variations, the devices, e.g., the hydrogel in the devices, may be configured to deliver one or more vulnerary agents to the wound. In some cases, a hydrogel in a device may be configured to deliver at least one vulnerary agent to the wound in response to an environmental stimulus. Such an environmental stimulus may for example be selected from the group consisting of temperature, pH, pK, electromagnetic radiation, electric field, ultrasound energy, and combinations thereof. In some variations, body temperature may stimulate or trigger delivery of at least one vulnerary agent from the hydrogel.

If a device, e.g., a hydrogel in a device, is configured to deliver one or more vulnerary agents to a wound, the vulnerary agent or agents may be any suitable agent, e.g., one or more vulnerary agents may be selected from the group consisting of growth factors, antifungals, antibiotics, antivirals, anti-parasitic agents, vitamins, pH buffering agents or compositions, enzymes (e.g., digestive enzymes such as papain), antibodies, and combinations thereof.

If a growth factor is used as a vulnerary agent, the growth factor may be selected from the group consisting of a platelet-derived growth factor (PDGF), an epidermal growth factor (EGF), a transforming growth factor (TGF), a basic fibroblast growth factor (FGF1), an acidic fibroblast growth factor (FGF2), a bone morphogenetic protein (BMP), hydroxylapatite, a hepatocyte growth factor (HGF), a vascular endothelial growth factor (VEGF), a granulocyte-colony stimulating factor (G-CSF), a granulocyte macrophage colony stimulating factor (GM-CSF), a neurotrophin, erythropoietin (EPO), thrombopoietin (TPO), and combinations thereof. The neurotrophin may for example be nerve growth factor (NGF) or neurotrophin-3 (NT-3). The transforming growth factor may in some cases be growth differentiation factor-8 (GDF-8) or growth differentiation factor-9 (GDF-9). Some variations of devices may be configured to deliver a vulnerary agent that comprises an engineered growth factor.

Methods for treating wounds are described here. These methods may comprise applying a pre-wetted IPN hydrogel to a wound. The IPN hydrogel used in the methods for treating wounds may comprise a first covalently cross-linked polymer network, and a second polymer network that has been cross-linked and/or polymerized in the presence of the first polymer network. The methods comprise controlling an amount of moisture in the wound as a function of time by tuning the properties of the first and second polymer networks in the IPN hydrogel. For example, the first polymer network may be selected to be a hydrophilic nonionic polymer that is cross-linked between ends. In some variations, the first polymer network may be selected to comprise a cross-linked PEG network. The second polymer network may be selected to be hydrophilic and/or ionizable, e.g., the second polymer network may comprise PAA.

Methods for making a wound dressing are described herein. These methods comprise forming a first hydrophilic polymer network that is cross-linked between ends, and forming a second polymer network by cross-linking and/or polymerizing precursor molecules in the presence of the first polymer network to form an interpenetrating network hydrogel. The methods further comprise pre-wetting the interpenetrating network hydrogel with a wetting agent to make a nonadhesive wound dressing that is configured to control an amount of moisture in a wound.

Some variations of these methods may comprise forming the first polymer network by cross-linking end-functionalized poly(ethylene glycol) (PEG) macromonomers. For example, the first polymer network may be formed by cross-linking diacrylate functionalized-PEG macromonomers. The end-functionalized PEG macromonomers may have a molecular weight of about 1 kDa to about 20 kDa, or about 3 kDa to about 8 kDa. Certain variations of the methods for making a wound dressing may comprise forming a second hydrophilic and/or ionizable polymer network, e.g., a second polymer network comprising poly(acrylic acid) (PAA).

The methods may comprise pre-wetting the hydrogel with any suitable wetting agent, e.g., saline or glycerol. Some methods may comprise pre-wetting the hydrogel to at least about 10% by weight with the wetting agent. Certain methods may comprise loading the hydrogel with one or more vulnerary agents, and configuring the hydrogel to deliver at least one vulnerary agent to a wound.

Kits for treating wounds are also described herein. In general, the kits comprise an IPN hydrogel, a wetting agent, and instructions for pre-wetting the hydrogel with the wetting agent. In some variations, the kits may further comprise one or more fasteners configured for securing the hydrogel to tissue proximal to the wound. The one or more fasteners may for example be selected from the group consisting of an adhesive, a suture, a tissue anchor, a staple, a clamp, and combinations thereof. Some kits may comprise one or more tools configured for securing, positioning, and/or removing the hydrogel from the wound. Certain kits may comprise multiple IPN hydrogels, wherein the multiple hydrogels are selected to be used sequentially and/or in combination, e.g., in various stages of wound healing.

Variations of these kits for treating wounds may comprise one or more vulnerary agents that may be delivered to the wound. In some of these kits, the vulnerary agents may be adapted to be loaded into the hydrogel and delivered by the hydrogel to the wound. If a kit comprises one or more vulnerary agents, at least one of the vulnerary agents may be selected from the group consisting of growth factors, antifungals, antibiotics, antivirals, anti-parasitic agents, vitamins, pH buffering agents or compositions, enzymes (e.g., digestive enzymes such as papain), antibodies, and combinations thereof.

Implants are also described herein. The implants comprise an IPN hydrogel, and are configured to be disposed under a tissue layer and to take up moisture at a gradual rate so that the implant expands the overlying tissue layer. In the implants, the hydrogel comprises a first polymer network that comprises a hydrophilic nonionic polymer that is covalently cross-linked between ends, and a second polymer network that has been cross-linked in the presence of the first polymer network. A hydrogel in the implant may be adapted to control a moisture take up rate, and/or an ultimate size and/or shape of implant. For example, a hydrogel used in the implants may be adapted to expand at a predetermined rate by taking up moisture from within a subject's body and/or from an external source. Alternatively or in addition, a hydrogel used in the implants may be adapted to have one or more predetermined expanded dimensions.

In a hydrogel used in an implant described herein, the first polymer network may comprise PEG or derivatives thereof. The PEG may comprise a segment having a molecular weight of about 1 kDa to about 20 kDa, or about 3 kDa to about 8 kDa between end cross-links. The second polymer network may be ionizable, and in some variations may comprise PAA.

The implants may be configured to be temporary implants that are removable after a desired amount of tissue expansion. In other variations, the implants may be configured to remain under the expanded tissue layer as long term implants. The hydrogel in some variations of implants may be configured to deliver a vulnerary agent to the subject.

Methods for expanding tissue using expandable implants are provided here. These methods comprise implanting an IPN hydrogel under a tissue layer. The hydrogel comprises a first covalently cross-linked polymer network and a second polymer network that has been cross-linked and/or polymerized in the presence of the first polymer network. The methods further comprise selecting the first and second polymer networks to control a dimension of the hydrogel as a function of time to expand the tissue layer. In some variations of the methods, the first polymer network may comprise a hydrophilic, nonionic polymer that is cross-linked between ends, e.g., the first polymer network may comprise PEG. In certain variations of the methods, the second polymer may be hydrophilic and/or ionizable, e.g., the second polymer network may comprise PAA.

Methods for making implants are described here. These methods comprise forming a first hydrophilic polymer network that is covalently cross-linked between ends, forming an IPN hydrogel by cross-linking and/or polymerizing a second polymer network in the presence of the first polymer network, configuring the hydrogel for implantation under tissue, and selecting the first polymer network and the second polymer network to control expansion of the tissue. In these methods, the first polymer network may be formed by cross-linking end-functionalized PEG macromonomers, e.g., diacrylate-functionalized PEG macromonomers. The second polymer network may be formed by cross-linking precursor molecules to form cross-linked PAA.

Kits comprising a set of implants are provided here. In these kits, at least one implant in the set comprises an IPN hydrogel that is configured to be placed under tissue of a subject and to progressively expand the overlying tissue. In these kits, the implants in the set may be configured to be placed in a serial manner and/or in combination under tissue of the subject to progressively expand the tissue. Some variations of kits may further comprise a wetting agent to be absorbed by a hydrogel.

### BRIEF DESCRIPTION OF THE FIGURES

The present invention together with its objectives and advantages will be understood by reading the following description in conjunction with the drawings, in which:

FIG. 1 illustrates an example of an IPN hydrogel comprising a first cross-linked polymer network fully interpenetrated by a second cross-linked polymer network.

FIGS. 2A-2C illustrate a variation of a method for synthesizing a fully interpenetrated IPN hydrogel.

FIGS. 3A-3D illustrate additional variations of methods for synthesizing IPN hydrogels.

FIGS. 4A-4D illustrate variations of substituted hydrophilic IPN hydrogels.

FIG. 5 illustrates time-dependent moisture uptake for examples of IPN hydrogels.

FIG. 6 illustrates stress-strain curves for variations of PEG/PAA IPN hydrogels.

FIGS. 7A-7B illustrate examples of wound dressings made using IPN hydrogels.

FIG. 8 illustrates a variation of a device configured to deliver a vulnerary agent to a wound and to absorb wound exudates.

FIGS. 9A-9B illustrate a side cross-sectional view of a variation of a device applied to a wound. In this particular example, the device has a first portion configured to absorb wound exudates and a second portion configured to deliver a vulnerary agent to the wound.

FIGS. 9C-9D illustrate cross-sectional and plan views, respectively, of another variation of a device applied to a wound.

FIGS. 9E-9F illustrate cross-sectional and plan views, respectively, of yet another variation of a device applied to a wound.

FIG. 10A illustrates a side cross-sectional view of a variation of a device in which the first absorptive portion and the second delivery portion each comprise multiple subportions in the form of bands. Bands of the first absorptive portion are alternated with bands of the second delivery portion. FIG. 10B illustrates a top plan view of the device shown in FIG. 10A.

FIG. 11 illustrates a side cross-sectional view of a variation of a device in which the spatial distribution of the absorptive portion and the delivery portion is not uniform across the device.

FIG. 12 illustrates a side cross-sectional view of variation of a device in which delivery subportions have varying concentrations of a vulnerary agent.

FIG. 13 illustrates a side cross-sectional view of a variation of a device in which the concentration of a vulnerary agent within a delivery portion or subportion is nonuniform.

FIGS. 14A-14B illustrate a variation of a device in which the first absorptive portion and the second delivery portion are arranged in a concentric manner. FIG. 14A is a cross-sectional side view and FIG. 14B is a top plan view.

FIG. 15A illustrates a side cross-sectional view of a variation of a device in which the first absorptive portion and the second delivery portion each comprise multiple concentric subportions. Subportions of the first absorptive portion are alternated with subportions of the second delivery portion. FIG. 15B illustrates a top plan view of the device shown in FIG. 15A.

FIG. 16A illustrates a side cross-sectional view of a variation of a device in which the first absorptive portion is contiguous and the second delivery portion comprises multiple noncontiguous subportions. FIG. 16B illustrates a top plan view of the device shown in FIG. 16A.

FIG. 17 shows a side cross-sectional view of a variation of a device having multiple layers.

FIG. 18 illustrates a side cross-sectional view of a variation of a device in which the vulnerary agent is attached to a component of the second delivery portion.

FIG. 19 illustrates a side cross-sectional view of a variation of a device comprising more than one delivery portion that is capable of delivering more than one vulnerary agent to the wound.

FIG. 20 illustrates a side cross-sectional view of a multilayer variation of a device comprising more than one delivery portion that is capable of delivering more than one vulnerary agent to the wound.

FIG. 21 shows a side cross-sectional view of a variation of a device having an airtight cover.

FIG. 22 illustrates diffusion processes between absorptive and delivery portions.

FIG. 23 shows a side cross-sectional view of a variation of a device having narrowly-spaced absorptive subportions interspersed with narrowly-spaced delivery subportions.

FIG. 24 illustrates a side cross-sectional view of a variation of a device having widely-spaced absorptive subportions interspersed with widely-spaced delivery subportions.

FIGS. 25A-25B illustrate a variation of a method for making a wound healing device using patterned irradiation. FIG. 25B shows a top plan view of a device made according to the method illustrated in FIG. 25A.

FIGS. 26A-26B illustrate another variation of a method for making a wound healing device using patterned irradiation.

### DETAILED DESCRIPTION

Described herein are devices that are configured to remove devitalized tissue and other wound exudates from a wound, and related methods and kits. These devices, methods and kits may be adapted to treat a variety of wounds over a range of timescales, and in some cases may be used to treat chronic wounds or wounds at high risk for infection.

In some variations, the devices and related methods and kits may utilize an interpenetrating polymer network (IPN) hydrogel. The IPN hydrogel may be surface treated so that the hydrogel is relatively nonadhesive to wound tissue in contact with the hydrogel, yet can absorb a substantial amount of moisture from the wound. For example, an IPN hydrogel may be pre-wetted to a predetermined degree with a wetting agent so as to be relatively nonadhesive to wound tissue. Certain properties of a pre-wetted hydrogel may be tuned to adjust a rate and/or an amount of moisture uptake by the hydrogel, e.g., the surface area of the hydrogel in contact with a wound, the volume or volume change of the hydrogel, the composition of one or more polymer networks making up the hydrogel, the wetting agent used to pre-wet a hydrogel, and any combination thereof may be varied to adjust the function of a hydrogel to control the amount of moisture in a wound over a period of time. Further, other physical properties of a hydrogel in either its swollen or relatively dry state may be tuned such as Young's modulus, flexural modulus, shear modulus, tensile strength, compressive strength, stiffness, and the like. Thus, wound dressings made from such pre-wetted hydrogels can be adapted to debride a variety of different wound types at different stages of healing over a variety of timescales, in some cases without requiring intervention in the form of frequent dressing changes and the like. In certain variations of the wound dressings and related methods and kits described herein, the IPN hydrogels may be adapted to deliver a vulnerary agent to the wound separately or in combination with wound debridement.

Also described herein are permanent or temporary expandable implants comprising hydrogels, e.g., IPN hydrogels, and related methods and kits. In general, the implants are configured to be positioned in a subject below a layer of tissue. The hydrogel in the implants may be selected to absorb moisture to expand at a desired gradual rate and/or to a desired ultimate shape and/or size to expand the overlying tissue. The tissue so expanded may be removed and used elsewhere, e.g., for a graft, or the tissue may be expanded to receive one or more other implants or prostheses. In some cases, the expandable implants described here may be left in a subject's body to remain as a long term or permanent implant. The physical properties and/or moisture content of a hydrogel used in the implants may be tuned so that the resulting implant has desired temporary or long term properties. For example, an IPN hydrogel used in the implants may be designed to have certain physical properties during and/or after taking up moisture, e.g., Young's modulus, flexural modulus, shear modulus, tensile strength, compressive strength, and/or stiffness, so as to improve the function of the implant. In certain variations, an IPN hydrogel used in an implant may be pre-wetted to allow the implant to take up moisture without substantially adhering to surrounding tissue. In other variations, an implant may adhere to surrounding tissue.

The term “gel” as used herein is meant to encompass a cross-linked polymer network having liquid molecules entrapped within the network. The term “hydrogel” is meant to encompass a cross-linked polymer network that can be swollen with water or aqueous solution to form a gel. Although hydrogels can be swollen with water, they are not generally soluble in water. A hydrogel can absorb many times its weight in water, e.g., about 5 times, about 10 times, or about 50 times, or about 100 times or more its weight in water. When a gel is capable of absorbing more than about 10 times its weight in liquid, it can be referred to as a superabsorbent gel. As used herein, “take up” or “absorb” is meant to encompass any form of absorption or adsorption, e.g., wicking, swelling, and the like.

The term “polymer network” is meant to encompass a three-dimensional network of polymer chains, which may or may not contain inter-chain cross-links. The term “macromonomer” as used herein is meant to encompass a macromolecule that has at least one end group that enables to it to function as a monomer in a final macromolecule, by contributing only a single monomeric unit to a chain of the final macromolecule. In some variations, a macromonomer may have two end groups that enable it to function as a monomer in a final macromolecule. A macromonomer may have any suitable molecular weight, e.g., about 50 to about 1×10⁶ Da, or about 50 to about 1×10⁵ Da, or about 50 to about 1×10⁴ Da, or about 50 to about 1000 Da.

The term “vulnerary agent” is meant to encompass any compound or composition that can aid in the healing of tissue and/or the promotion of tissue growth. Vulnerary agents may also prevent or reduce scar formation during healing. Vulnerary agents may modify the wound environment to make the environment more conducive to healing, e.g., by decreasing acidity. Certain vulnerary agents may aid in the removal of cell debris, e.g., vulnerary agents may comprise digestive enzymes such as papain. Vulnerary agents may include, for example, growth factors, antibodies, antiseptics or antimicrobials such as antibiotics, antifungals, antivirals, anti-parasitic agents, vitamins, buffer agents, enzymes, and combinations thereof. In some variations, vulnerary agents may include therapeutic proteins, or DNA molecules that encode for therapeutic proteins.

### I. Devices and Implants

Devices for assisting in wound healing may thus comprise an IPN hydrogel that comprises a first cross-linked (e.g., covalently cross-linked) polymer network and a second polymer network that has been cross-linked and/or polymerized in the presence of the first polymer network. The first polymer network may comprise a hydrophilic, nonionic, end cross-linked polymer network, e.g., an end cross-linked polymer network formed by cross-linking end-functionalized PEG as described above. The second polymer network may be a hydrophilic and/or ionizable polymer network, e.g., a cross-linked PAA network.

A hydrogel used in the absorptive devices and implants described herein may comprise synthetic or naturally-occurring hydrophilic polymers, or a combination of synthetic and natural polymers. Further, absorptive hydrogels may comprise homopolymers, copolymers, including block copolymers, blends, or interpenetrating networks of two or more polymers. Examples of suitable hydrophilic synthetic polymers that form hydrogels include poly(hydroxyethyl methacrylate) (PHEMA), poly(ethylene glycol) (PEG), poly(vinyl alcohol) (PVA), poly(acrylic acid) (PAA), poly(methacrylic acid) (PMAA), polyacrylamide (PAAm), and derivatives, copolymers (including block copolymers), and blends thereof. Examples of polymers derived from natural sources that can be incorporated into the absorptive hydrogels include collagen, hyaluronic acid, fibrin, alginate, poly(glutamic acid), chitosan, poly(lysine), gelatin, and agarose, and blends and copolymers thereof. Hydrogels used in devices described herein should be biocompatible, i.e., non-toxic and non-immunogenic to humans. For example, PEG is inert to many biological molecules, such as proteins. Hydrogels made from PEG are both non-toxic and non-immunogenic to humans, and have already been approved by the U.S. Food and Drug Administration for some clinical uses. PHEMA, PVA, and PNIPAAm hydrogels are also non-toxic and non-immunogenic to humans.

In general, the hydrogel polymer networks used in the devices, wound dressings and/or implants described herein may be covalently (chemically) or physically cross-linked. The hydrogels used in the devices, wound dressings and/or implants may contain a sufficient degree of cross-linking to render the hydrogel substantially insoluble in water. The degree of cross-linking in the hydrogels may be varied, and may be modified to tune desired chemical and/or physical hydrogel properties. The cross-linking interactions between polymer chains in a hydrogel may be either covalent or non-covalent. Cross-linking to form covalent bonds may be accomplished by any suitable method, e.g., through the use of cross-linkable functional groups (end groups and or side chains), chemical cross-linking agents, photo-initiated cross-linking, radiation-induced cross-linking, e.g., by electron beam, thermally-induced cross-linking, or combinations thereof. Non-limiting examples of physical or non-covalent cross-linking mechanisms include hydrogen bonding and ionic or electrostatic interactions. Alternatively or in addition to varying the cross-linking density in one or more polymer networks, the mechanical and physical properties of a hydrogel may be tuned by adjusting the molecular weight of segments between cross-links in a polymer network in the hydrogel.

In some variations of the devices and/or implants, a hydrogel may be able to absorb at least about 50% to about 100% of its weight in water. In certain variations, a hydrogel may absorb at least about 2 times, at least about 5 times, at least about 10 times, at least about 15 times, at least about 100 times, or more its weight in water. The molecular structure of highly absorbent (“superabsorbent”) hydrogels may comprise at least about one-quarter hydrophilic groups. As will be discussed in more detail below, the amount and/or rate of moisture absorption by a hydrogel may be tuned depending on its application as a wound dressing and/or implant. Non-limiting examples of highly absorbent hydrogels include cross-linked polyacrylamides, cross-linked sulfonated polystyrenes, cross-linked poly(alkylene oxides) or derivatives, copolymers, or blends thereof. In some variations, a hydrogel may include a hygroscopic substance to increase its ability to absorb wound exudates.

As described above, some variations of the wound dressing devices and/or implants described herein may comprise a three-dimensional IPN hydrogel. An IPN hydrogel comprises at least two polymer networks, wherein one polymer network interpenetrates the volume occupied by another polymer network. As used herein “IPN” may refer to either a semi-interpenetrating polymer network (SIPN), where the two polymer networks are physically entangled but one of the polymer networks is not irreversibly entwined with the other polymer networks by the formation of covalent bonds, or a fully interpenetrating polymer network (IPN), where the two polymer networks are irreversibly entwined with each other by the formation of covalent bonds. In certain variations of hydrogels, two polymer networks may interact in addition to being entangled, e.g., covalent or non-covalent interactions such as hydrogen bonding may be present between groups on two different polymer networks. In some variations, the IPN hydrogel may comprise more than two polymeric components, e.g., a precursor for a third polymer can be dispersed through an IPN formed by the first two polymers. The third polymer can then be polymerized and/or cross-linked in situ to form a third polymer network.

The first polymer network of an IPN hydrogel may be based on PEG, PHEMA, PVA, PAA, collagen, hyaluronan hydrogel, or derivatives thereof. PEG is biocompatible, soluble in aqueous solution and can be synthesized in a range of molecular weights. The second polymer network of an IPN hydrogel may be based on PAA, PAAm, PNIPAAm, PMAA, poly(2-acrylamido-2-methylpropanesulfonic acid) (PAMPS), poly(2-hydroxyethyl methacrylate), poly(2-hydroxyethyl acrylate), or derivates thereof.

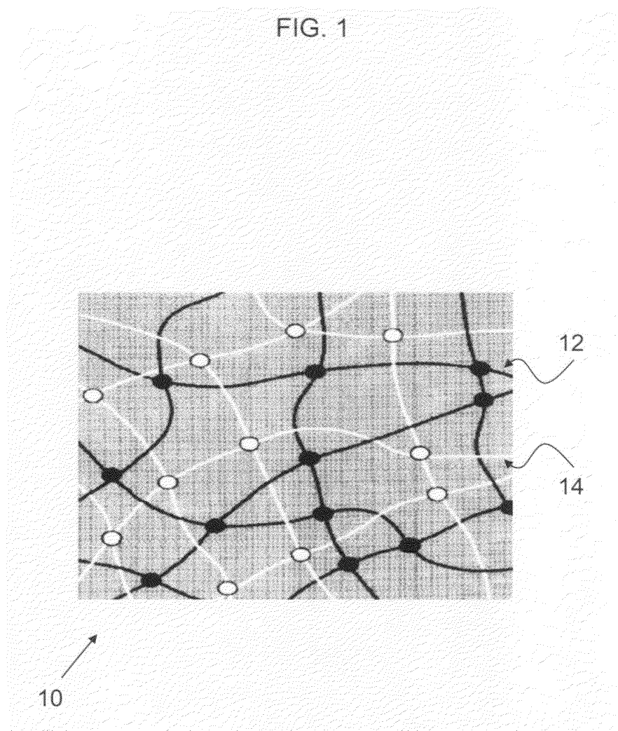

Referring now to FIG. 1, a schematic illustration of an IPN hydrogel that may be used in the wound dressings and implants described herein is shown. There, the IPN hydrogel 10 comprises a first cross-linked polymer network 12 that is fully interpenetrated with a second cross-linked polymer network 14. If the two polymer networks are irreversibly intertwined by the formation of covalent bonds in the networks as shown, each network may retain its physical properties relatively independently of the other network, allowing for tuning of physical properties of the hydrogel 10 by varying the proportion and nature of the first polymer network 12 and the second polymer network 14.

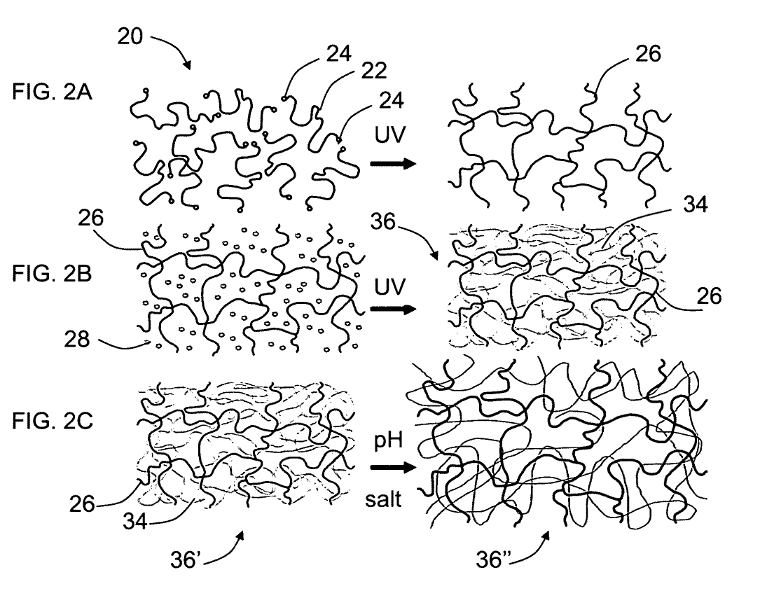

An example of a method for synthesizing an IPN hydrogel is shown in FIGS. 2A-2C. In FIG. 2A, the formation of the first network of the IPN hydrogel is illustrated. There, macromonomers 22 in an aqueous solution 20 are cross-linked via the functionalized end groups 24 of the macromonomers to form a first network 26 in aqueous solution. In this particular variation, the macromonomers 22 have functionalized end groups 24 on both ends, to form a highly cross-linked end-linked network 26. At this stage, the first network 26 is not soluble in aqueous solution, but is swollen with aqueous solution. As shown in FIG. 2A, UV radiation may be used to induce cross-linking. In other variations, other means may be used to induce cross-linking to form the first polymer network, e.g., thermal cross-linking or radiation cross-linking. Referring now to FIG. 2B, the first network 26 can be swollen with monomers 28 in water, optionally along with one or more photoinitiators (not shown) and one or more cross-linking agents (not shown). The monomers 28 may be any suitable monomers, but in some variations they may be hydrophilic and/or ionizable. The monomers 28 shown on the left in FIG. 2B may be cross-linked (e.g., by UV radiation) around and between the first network 26 to form the second network 34 that is irreversibly interpenetrated with the first network 26 to yield the IPN 36. Referring now to FIG. 2C, the IPN 36 can be subsequently exposed to solution and swelled to form a swollen network 36′, e.g., by exposure to a solution having a certain pH and/or salt content to an equilibrium point. In some cases, the swollen IPN 36′ may have a higher Young's modulus than the relatively non-swollen IPN 36, e.g., if the strain induced by swelling results in an increase in density of physical cross-links between the first and second polymer networks in the swollen IPN 36′.

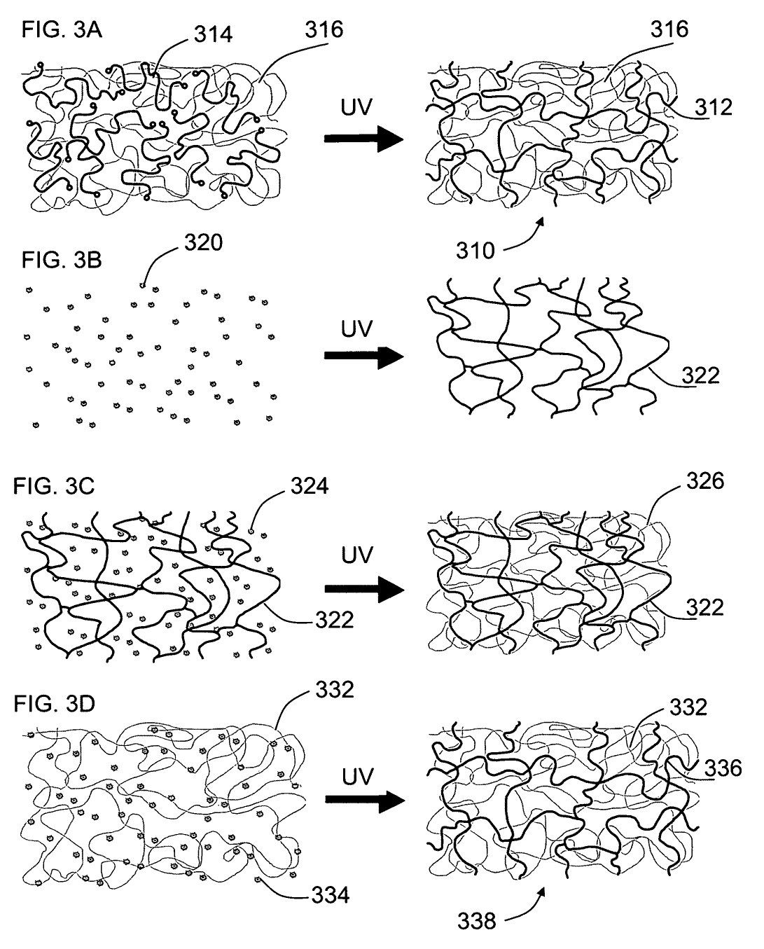

Referring to FIGS. 3A-3D, examples of various types of IPNs and methods for forming the IPNs are shown. In FIG. 3A, a second polymer network 312 is formed by cross-linking of a functionalized macromonomer 314 or monomer in the presence of a first non-cross-linked polymer network 316. If the second polymer 316 does not chemically cross-link, then the resulting IPN hydrogel 310 is a semi-interpenetrating network (SIPN). That is, although the first non-cross-linked polymer network 316 is physically entwined with the second cross-linked polymer network 312, the two networks are not interlocked as only one of the networks is cross-linked. Referring now to FIGS. 3B-3C, a monomer 320 is first cross-linked, e.g., by exposure to UV light, to form a first network 322. The first network 322 can be swollen with precursor molecules 324, e.g., monomers (and optionally a cross-linking agent (not shown) and/or a photoinitiator (not shown)), that can be cross-linked and/or polymerized to form a second network 326. Cross-linking and/or polymerization of the precursor molecules 324, e.g., by exposure to UV light, leads to the formation of an IPN hydrogel 328. If the second network 326 is not cross-linked, the IPN hydrogel 328 will be a SIPN. Referring now to FIG. 3D, a first non-cross-linked polymer network 332 is mixed in solution with precursor molecules 334, e.g., monomers and optionally one or more photoinitiators and/or cross-linking agents. The precursor molecules 334 can be cross-linked, e.g., by exposure to UV light, to form a second polymer network 336 in the presence of the first polymer network 336 to form an IPN 338. Here again, if the first non-cross-linked polymer network 332 does not cross-link so as to interlock with the second polymer network, then the IPN 338 is a SIPN.

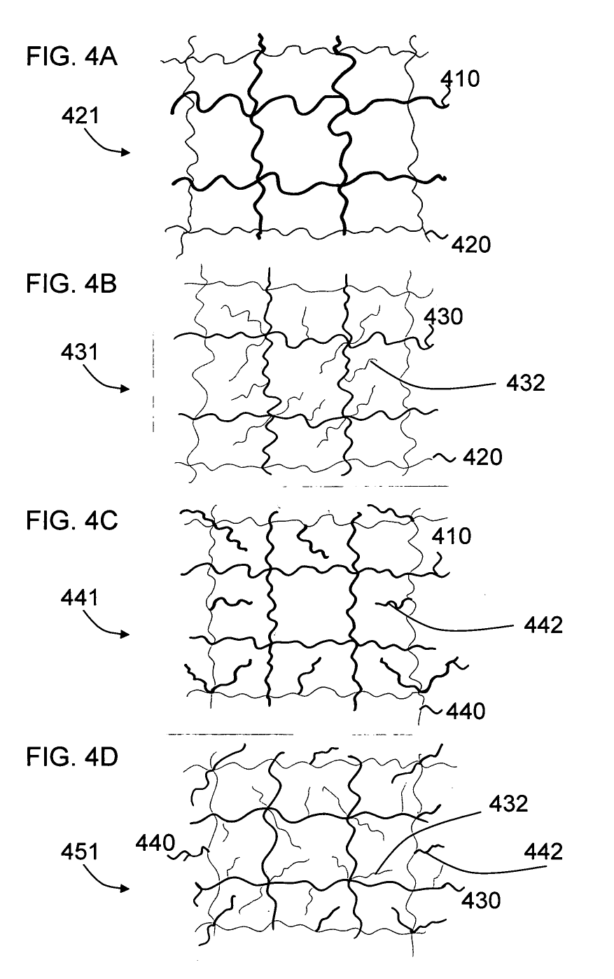

Referring to FIGS. 4A-4D, a variation of an IPN that may be used in the implants or wound dressing devices described herein is shown in which at least one of the polymer networks in the IPN is grafted with a hydrophilic polymer or side group. FIG. 4A illustrates an IPN 421 comprising a first polymer network 420 and a second polymer network 410. In the variation shown in FIG. 4B, the IPN 431 comprises the first polymer network 420 and a second polymer network 430 that has been grafted with a hydrophilic side group 432. In the variation shown in FIG. 4C, an IPN 441 is shown that comprises the original second polymer network 410 as shown in FIG. 4A, but a first polymer network 440 that has been grafted with hydrophilic side groups 442. In the variation shown in FIG. 4D, an IPN hydrogel 451 is shown that comprises a first polymer network 440 that is grafted with hydrophilic groups 442, and a second polymer network 430 that is grafted with hydrophilic groups 432. In each of the examples illustrated in FIGS. 4A-4D, a grafted polymer may be formed by any known technique, e.g., by copolymerizing first and second polymer precursors in solution in a ratio that results predominantly in the formation of one of the first and second polymers, but included grafted sections or side chains of the other of the first and second polymers.

Some variations of the wound dressings and implants described here may comprise an IPN hydrogel that comprises a pH neutral, cross-linked hydrophilic network of macromonomers cross-linked between ends. As will be discussed in more detail below, this first cross-linked polymer network may, for example, comprise end cross-linked poly(ethylene glycol) (PEG) macromonomers with defined molecular weights or molecular weight ranges. The second polymer network of the IPN may also be hydrophilic, and in some cases may be ionizable. The second polymer network may be polymerized and/or cross-linked in the presence of the first polymer network. If the second polymer network is cross-linked in the presence of the first polymer network, the second network may be fully interpenetrated with the first cross-linked network. The second polymer network may comprise poly(acrylic acid) (PAA). Some examples of IPN hydrogels that can be used in the devices and implants described herein, and in particular IPN hydrogels based on PEG/PAA, have been previously described, e.g., in “Strain-Hardened Interpenetrating Polymer Network Hydrogel,” U.S. patent application Ser. No. 12/070,336, filed Feb. 15, 2008, “Interpenetrating Polymer Network Hydrogel Corneal Prosthesis,” U.S. Patent Publication No. 2007/0179605, published Aug. 2, 2007, “Artificial Corneal Implant,” U.S. Patent Publication No. 2006/0083773, published Apr. 20, 2006, “Artificial Cornea,” U.S. Patent Publication No. 2006/0287721, published Dec. 21, 2006, “Intraocular Lens Implant,” U.S. Patent Publication No. 2007/0233240, published Oct. 4, 2007, and “Interpenetrating Polymer Network Hydrogel Contact Lenses,” U.S. Patent Publication No. 2007/0126982, published Jun. 7, 2007, each of which is hereby incorporated by reference in its entirety.

Any suitable precursor, e.g., monomer or macromonomer, may be used to form the first network in the IPN hydrogel used in the devices and implants described herein. As stated above, polyethylene glycol (PEG) macromonomers may be used to form the first network. PEG is biocompatible, soluble in aqueous solution, and can be synthesized in a range of molecular weights and/or with a variety of side chains. A hydroxyl end group or side group on a PEG macromonomer can be functionalized to become a cross-linkable end group, e.g., a UV cross-linkable end group. For example, end group or side group cross-linkable functionalities include acrylates, e.g., to form PEG-diacrylate, methacrylate, e.g., to form PEG-dimethacrylate, allyl ethers, e.g., to form diallyl ethers, vinyl, e.g., to form divinyls, acrylamides, e.g., to form PEG-diacrylamide, methylacrylamides, e.g., to form PEG-dimethylacrylamide. Other types of polymers or macromonomers may be functionalized with similar end groups or side groups, e.g., polycarbonate, poly(N-vinyl pyrrolidone), polyurethane, poly(vinyl alcohol), polysaccharides such as dextran, collagen, and derivatives and combinations thereof. Thus, the first network may comprise functionalized PEG macromonomers copolymerized and/or cross-linked with other types of polymers and macromonomers. For example, the first network may be copolymerized and/or cross-linked with polymers comprising acrylamide such as hydroxyethyl acrylamide and N-isopropylacrylamide, polyurethane, 2-hydroxyethyl methacrylate, polycarbonate, 2-hydroxylethyl acrylate, and derivatives thereof.

The precursor to the second network in the IPN hydrogel may be an ionizable monomer, and in some cases may be capable of being negatively charged (anionic). For example, acrylic acid monomers in aqueous solution may be used as precursor molecules to the second polymer network. In other variations, precursors to a second polymer network in the IPN hydrogel may comprise negatively charged carboxylic acid groups or sulfonic acid groups, such as methacrylic acid, 2-acrylamido-2-methylpropanesulfonic acid, hyaluronic acid, heparin sulfate, chondroitin sulfate, or derivatives or combinations thereof. In certain variations, the precursors to the second polymer network may be capable of being positively charged (cationic). In still other variations, the precursor molecules to the second polymer network may be nonionic, e.g., acrylamide, methacrylamide, N-hydroxylethyl acrylamide, N-isopropylacrylamide, methylmethacrylate, N-vinyl pyrrolidone, 2-hydroxyethyl methacrylate, 2-hydroxyethyl acrylate, or derivatives or combinations thereof. Hydrophilic monomers that are used as precursor molecules to the second polymer network may be copolymerized with less hydrophilic monomers such as methylmethacrylate. The second polymer network may also comprise proteins or polypeptides such as collagen, hyaluronic acid, chitosan, or derivatives thereof.

In certain variations, one or more cross-linking agents may be used to form cross-links in the first and/or second polymer networks of the IPN hydrogels. Non-limiting examples of cross-linking agents include ethylene glycol dimethacrylate, ethylene glycol diacrylate, diethylene glycol dimethacrylate, diethylene glycol diacrylate, triethylene glycol dimethacrylate, triethylene glycol diacrylate, tetraethylene glycol dimethacrylate, tetraethylene glycol diacrylate, polyethylene glycol dimethacrylate, polyethylene glycol diacrylate, methylene bisacrylamide, N,N′-(1,2-dihydroxyethylene) biascrylamide, and derivatives and combinations thereof. In the case that photoinitiated, e.g., UV-initiated, cross-linking is used to form the first and/or second polymer networks, one or more photoinitiator may be used. Non-limiting examples of photoinitiators that may be used include 2-hydroxy-2-methylpropiophenone, 2,2-dimethoxy-2-phenylacetophenone, and 2-hydroxy-1-[4-(2-hydroxyethoxy) phenyl]-2-methyl-1-propanone.

In a hydrogel network made from a first network comprising PEG and a second network comprising PAA, hydrogen bonding complexes between the ether group on the PEG and carboxyl groups on PAA can form inter-network hydrogen bonding. The PAA network is ionizable, having a pKa=4.7, whereas the PEG is not ionizable. Therefore, if the hydrogel is exposed to a solution having a pH higher than about 4.7, the PAA will lose a proton to the solvent and become negatively charged and swell. At a pH lower than about 4.7, the PAA network may become protonated and contract in the presence of the solution.

An IPN hydrogel that may be used in a wound dressing device and/or in an expandable implant may be formed by a two step synthesis. A first hydrophilic cross-linked end-linked polymer network may be formed by free radical polymerization, e.g., UV-initiated free radical polymerization, of PEG macromonomers with functionalized cross-linkable end groups. A solution, e.g., in phosphate buffered saline (PBS), water, or an appropriate organic solvent, of purified PEG macromonomers with functionalized end groups may be prepared. One or more UV-sensitive photoinitiators such as 2-hydroxy-2-methylpropiophenone, 2,2-dimethoxy-2-phenylacetophenone, or 2-hydroxy-1-[4-(2-hydroxyethoxy) phenyl]-2-methyl-1-propanone may be added to the solution. Non-limiting examples of PEG-based macromonomers with cross-linkable functionalized end groups that may be used to form the first polymer network include PEG-diacrylate, PEG-dimethacrylate, PEG-diacrylamide, and PEG-diallyl ether. As stated above, cross-linking of the functionalized macromonomers may be accomplished by means other than UV initiated cross-linking, e.g., thermal cross-linking may be used. If UV irradiation is used to initiate cross-linking to form the first polymer network of the IPN hydrogel, the solution containing the functionalized PEG-based macromonomer may be contained by a mold that is at least partially transparent to UV light so that the cross-linking may be accomplished in the mold. Upon cross-linking, the cross-linked PEG-based end-linked network becomes insoluble in aqueous solution and forms a gel.

The second network in the IPN hydrogels may be created in the presence of the first network prepared as described above. The first network in its gel form may be immersed in a solution containing a precursor molecule for the second polymer network. For example, the first network may be immersed in a solution of about 10% (v/v) to about 100% (v/v) of acrylic acid. The solution may contain one or more photoinitiators and one or more cross-linking agents, e.g., from about 0.1% (v/v) to about 10% (v/v) triethylene glycol dimethacrylate (TEGDMA), triethylene glycol divinyl ether, N—N-methylene bisacrylamide, and N—N′-(1,2,-dihydroxyethylene)bisacrylamide, which may each be used alone, or which may be used in combinations with each other. The solution may be allowed to sit at room temperature to allow the first network gel to become equilibrated and swollen with the precursors to the second network, e.g., for about 24 hours. The solution comprising the first swollen network may then be cross-linked, e.g., by exposures to UV radiation, to form a second cross-linked polymer network in the presence of the first cross-linked polymer network to form a fully interpenetrated IPN hydrogel. In general, the molar ratio of the PEG-based macromonomers used to form the first network to the precursor molecules for the second network may be about 1:1, about 1:10, about 1:100, about 1:1000, about 1:2000, about 1:3000, about 1:4000, about 1:5000, or even more. In the IPN hydrogel, the first network may be present at a weight that is about 1/10, about the same as, or about 10 times higher than the weight of the second network. Physical characteristics of the IPN hydrogels as a function of moisture content may be varied by independently controlling the type and molecular weights of the precursors to the first and second polymer networks, and the conditions of IPN formation, such as precursor concentration in solution and cross-linking density (e.g., as determined by UV irradiation time for UV initiated cross-linking).

For PEG/PAA IPN hydrogels made from PEG-based macromonomers and PAA, PEG-based macromonomers of any suitable molecular weight may be used. For example, PEG-based macromonomers (e.g., PEG-DA, PEG-dimethacrylate, PEG-diacrylamide, PEG-diallyl ether and combinations thereof having a molecular weight from about 300 Da to about 20 kDa, or about 1 kDa to about 10 kDa, or about 3 kDa to about 8 kDa, or about 5 kDa to about 8 kDa, may be used. For example, in some variations, PEG-DA macromonomers having a molecular weight from about 275 Da to about 20 kDa, or about 1 kDa to about 10 kDa, or about 3 kDa to about 8 kDa, or about 5 kDa to about 8 kDa may be used. In general, the molecular weight of the PEG in the first polymer network may be used to tune the permeability, tensile properties such as Young's modulus, flexural modulus, tensile strength, shear modulus, shear strength, stiffness, compressive strength, and other mechanical and physical properties of the IPN hydrogel. For example, a higher molecular weight PEG-based macromonomer may generally lead to improved mechanical properties of the IPN hydrogel. In some variations, PEG-DA macromonomers having a molecular weight of about 7 kDa or 8 kDa may be used to reduce brittleness. Further, a higher molecular weight PEG-based macromonomer may lead to improved moisture absorption of the IPN hydrogel. Further, a molar ratio between the PEG macromonomer and a monomeric component of the PAA may be varied, e.g., from about 1:100, about 1:200, about 1:400, about 1:600, about 1:800, about 1:1000, about 1:1200, about 1:1400, about 1:1600, about 1:1800 or about 1:2000.

The wound healing devices may be used generally to control the moisture content of a variety of wound environments, and at various stages of healing. In general, a moist environment may speed healing and decrease scarring. However, excess moisture may lead to wound maceration, especially in the presence of pressure on the skin. Different types of wounds may heal better in the presence of different amounts of moisture. The rate of moisture uptake and/or an equilibrium moisture content of an IPN hydrogel used in the wound healing devices may be tuned by adjusting the composition and/or cross-linking density of the first and/or second polymer networks in the IPN hydrogel. Further, the relative proportions of the first and second polymers in the IPN hydrogel may be varied to adjust the hydrogel for treating a variety of wound healing conditions. For example, the rate of moisture uptake and equilibrium moisture content may be tuned by varying the composition of the second polymer network. If a very hydrophilic polymer such as PAA is used as the second polymer network, and the PAA is present in a relatively large proportion, the IPN hydrogel may be used to absorb relatively high quantities of wound exudates quickly. Further, a first polymer network made from relatively high molecular weight PEG-based macromonomers may be able to absorb higher quantities of moisture. Such variations of highly absorptive IPN hydrogels may be used in wounds that are producing high volumes of wound exudates. Under other circumstances, e.g., for wounds requiring lower rates of moisture uptake, a different IPN hydrogel may be used to control the moisture of the wound environment, e.g., an IPN hydrogel with less hydrophilic components in the first and/or second polymer network may be used so that the IPN hydrogel does not absorb excess moisture out of the subject's tissue but still may control the moisture of the wound to an acceptable level to facilitate healing. In some instances, a single wound healing hydrogel or device may be suitable to control the moisture content of the wound throughout various stages of healing, whereas in other instances, one wound healing hydrogel may be used in one stage of healing, and another wound healing hydrogel may be used in another stage of healing. For example, a wound healing hydrogel that is capable of absorbing relatively large amounts of moisture in a short period of time may be used in early healing phases, and a hydrogel that absorbs moisture more slowly may be used in later healing phases.

The expandable implants described here comprise an IPN hydrogel that may be configured to be placed under a layer of tissue, and to expand gradually or progressively by taking up moisture so as to expand the overlying tissue. The IPN hydrogel may be configured to take up moisture from the subject's body, and/or from an external source. For example, the IPN hydrogel may be periodically injected with solution, e.g., a saline solution, or be configured to gradually take up solution from an external reservoir that may or may not be attached to a subject's body. In some applications, the expanded tissue may be harvested and used elsewhere, e.g., as grafts. In some cases, the IPN hydrogel may be removed after a desired amount of tissue expansion, and the volume created by the expansion of the tissue may receive another implant, e.g., a reconstructive prosthesis. In other cases, the IPN hydrogel in the expandable implant may remain as a temporary or permanent implant under the expanded tissue.

FIG. 5 illustrates examples of time-dependent moisture uptake of examples of PEG and PEG/PAA IPN hydrogels. The time-dependent water content of an individual PEG polymer network as well as PEG/PAA IPN hydrogels comprising 25 wt % PAA and 50 wt % PAA were measured by drying the original hydrogels in a desiccator, placing the hydrogels in deionized water, and weighing the hydrogels at designated time intervals. As shown, the hydrogel comprising an individual PEG polymer network reaches an equilibrium moisture content first, whereas the IPN hydrogels reached equilibrium slightly slower. In all three of these example hydrogels, the majority of the moisture uptake occurred in less than about 5 minutes. However, other hydrogels may be used in accordance with the description provided herein that absorb moisture over different timescales, e.g., longer timescales.

The IPN hydrogels described herein may be configured to absorb a desired amount of moisture in an equilibrium state. In certain variations, the IPN hydrogels may absorb about 15% to about 95% moisture, or about 20% to about 90%, or about 50% to about 90% (all percentages by weight). The amount of moisture absorbed may depend on the pH of the solution being absorbed if at least one of the polymer networks in the hydrogels is ionizable. For example, if ionizable PAA or a derivative thereof is used as the second polymer network, the amount of moisture taken up by the IPN hydrogel will depend on the pH of the environment of the PAA. As shown below, an IPN hydrogel comprising PAA may take up more moisture in an acidic environment than in a pH neutral environment. The amount of moisture absorbed may depend on the ionic strength of the solution. For example, PEG/PAA hydrogels may absorb less water from a swelling solution having a higher salt concentration. Increased salt in the swelling solution screens negative charges in the ionized PAA network, reducing electrostatic repulsion and in turn the swelling of the PAA network.

The moisture content of a hydrogel may be evaluated by a ratio of the mass of the hydrogel in a dry state to its mass in a swollen state. Referring now to Table 1, the swelling behavior of individual polymer networks of PAA, PEG, and IPN hydrogels of PEG/PAA in water were investigated. To simulate swelling under physiological conditions, the hydrogels were also placed in PBS, pH=7.4, and ionic strength (I)=0.15. The equilibrium water content (%) and swelling ratio (q) of IPN hydrogels comprising a first polymer network formed from PEG macromonomers having molecular weights (M.W.) of 3.4 kDa, 4.6 kDa, and 8 kDA that are cross-linked between ends are shown. As shown in Table 1, the water content of the PEG (M.W.=3.4 kDa)/PAA swells to 70% water content at equilibrium in PBS, whereas PEG (M.W.=4.6 kDa)/PAA swells to 77% and PEG (M.W.=8.0 kDa)/PAA swells to 90%.

TABLE 1
Equilibrium moisture content (wt. %) and swelling ratio (q*) of PEG and PAA
single networks and PEG/PAA IPN hydrogels under various swelling conditions

| Specimen | Conditions | Water Content (wt. %) | Swelling ratio (q) |
|---|---|---|---|
| PAA | dH₂O** | 90.0 +/− 1.7 | 10.0 |
| PAA | pH 7.4, I = 0.15 | 95.5 +/0 1.7 | 22.1 |
| PEG (M.W. = 3.4 kDa) | dH₂O | 79.3 +/− 2.1 | 4.8 |
| PEG (M.W. = 3.4 kDa)/PAA | dH₂O | 56.3 +/− 3.1 | 2.4 |
| PEG (M.W. = 3.4 kDa)/PAA | pH = 7.4, I-0.15 | 68.7 +/− 1.6 | 3.2 |
| PEG (M.W. = 4.6 kDa) | dH₂0 | 84.5 +/− 0.4 | 6.5 |
| PEG (M.W. = 4.6 kDa)/PAA | dH₂O | 57.0 +/− 0.6 | 6.5 |
| PEG (M.W. = 4.6 kDa)/PAA | pH = 7.4, I = 0.15 | 77.0 +/− 1.2 | 4.4 |
| PEG (M.W. = 8.0 kDa) | dH₂O | 90.5 +/− 1.2 | 10.5 |
| PEG (M.W. = 8.0 kDa)/PAA | dH₂O | 80.2 +/− 1.5 | 5.1 |
| PEG (M.W. = 8.0 kDa)/PAA | pH = 7.4, I = 0.15 | 90.9 +/− 0.1 | 11.0 |

*q = Ws/Wd = mass of swollen polymer/mass of dry polymer
**dH₂O = deionized water

As stated above, the moisture uptake properties of the IPN hydrogels described herein may be varied by varying the composition of the second polymer network. Referring now to Table 2, the equilibrium water content of PEG (M.W.=8.0 kDa)-based IPN hydrogels as a function of acrylic acid (AA) monomer used in the formation of the second polymer network that is cross-linked in the presence of the first PEG-based cross-linked network. The IPN hydrogels were formed using an initial concentration of 50 vol. % PEG macromonomers in aqueous solution. Table 3 below provides the relative percentages of PEG and PAA in which the dry hydrogels prepared with varying concentrations of AA monomer to form the second polymer network in the IPN hydrogel.

TABLE 2
Equilibrium Moisture Content of PEG (M.W. =
8.0 kDa)/PAA IPN hydrogels made with varying
concentrations of acrylic acid (AA) monomer

| Concentration of AA | Equilibrium moisture |
|---|---|
| monomer in aqueous | content of PEG/PAA |
| solution (vol. %) | IPN hydrogel (wt. %) |
| 30% | 99% |
| 40% | 91% |
| 50% | 83% |

TABLE 3
IPN hydrogels prepared with varying concentrations
of acrylic acid (AA) monomer

| | | | Ratio of wt. % |
|---|---|---|---|
| Concentration of AA | Wt. % PEG | Wt. % PAA | PEG/wt. % PAA |
| monomer in aqueous | in dry IPN | in dry IPN | in dry IPN |
| solution (vol. %) | hydrogel | hydrogel | hydrogel |
| 30% | 23.5% | 76.5% | 0.30 |
| 40% | 17.5% | 82.5% | 0.20 |
| 50% | 13.0% | 87.0% | 0.15 |

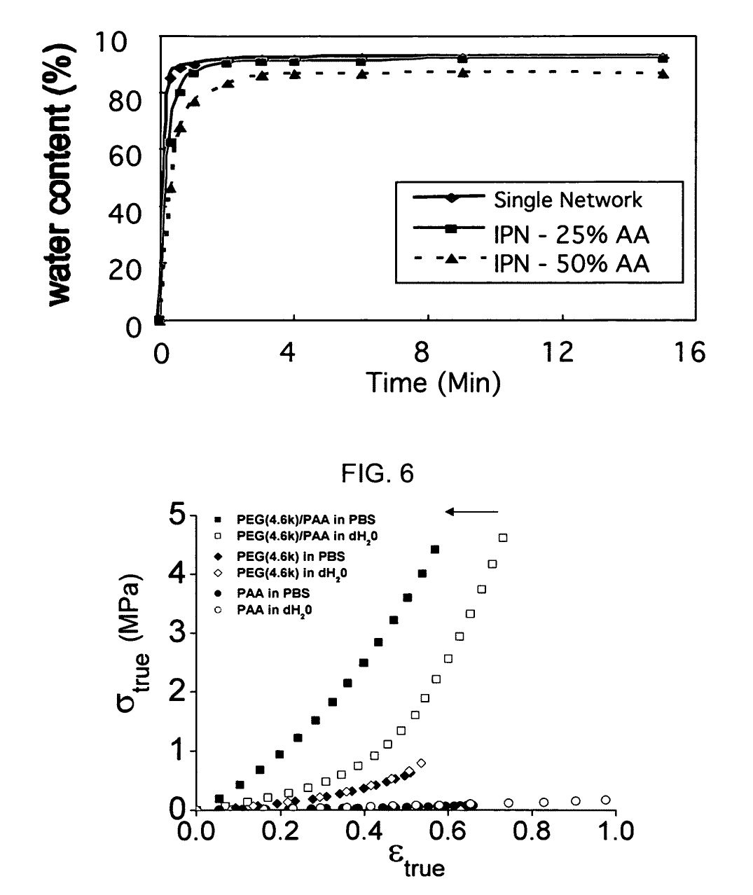

Cross-linking of an end-functionalized PEG-based macromonomer with lower molecular weight can lead to a more densely cross-linked PEG network. Referring now to FIG. 6, the stress (σₜᵣᵤₑ (MPa)) vs. strain (εₜᵣᵤₑ) for PEG (M.W.=4.6 kDa)/PAA hydrogels, as well as individual polymer networks made from PEG (M.W.=4.6 kDa) and PAA are shown. For each polymer network, stress vs. strain is shown for the network swollen to equilibrium with deionized water (pH=5.5, salt-free), and PBS (pH=7.4, ionic strength=0.15). There, it is seen that the stress-strain profile for the swollen polymer networks shifts upward when the PEG/PAA IPN hydrogel is swollen with PBS instead of water. The increase in the pH to 7.4 from 5.5, as well as the addition of salt causes the ionizable PAA network to swell disproportionately relative to the nonionic PEG network, resulting in the increase in modulus for the PEG/PAA hydrogel swollen in an environment simulating physiological conditions. As discussed in U.S. patent application Ser. No. 12/070,336, “Strain-Hardened Interpenetrating Polymer Network Hydrogel,” filed Feb. 15, 2008, which has already been incorporated herein by reference in its entirety, similar results are observed for PEG/PAA IPN hydrogels prepared from a PEG-diacrylamide first network and a PAA second network cross-linked using N,N′-(1,2-dihydroxyethylene) bisacrylamide. This phenomenon can be extended to tune the physical properties such as modulus by varying the relative concentration of the PAA and PEG in the IPN hydrogels, and tuning the cross-linking density in the PEG network and in the PAA network.

As stated above, other mechanical properties of an IPN hydrogel used in either a wound healing application or as an expandable implant may be varied by adjusting the composition and/or cross-linking density of the first and/or second polymer networks in the IPN hydrogel. For example, the Young's modulus of IPN hydrogels described herein even when swollen with moisture may be relatively high, e.g. greater than about 100 kPa, for example, from about 100 kPa to about 2 MPa, or about 100 kPa to about 5 MPa, or about 100 kPa to about 10 MPa, or even higher. The tensile strength of the IPN hydrogels even when swollen may be also greater than about 100 kPa, e.g., from about 100 kPa to about 2 MPa, or about 100 kPa to about 5 MPa, or about 100 kPa to about 10 MPa, or even higher. Further, the unconfined compression strength of the IPN hydrogels may be greater than about 100 kPa, e.g., e.g., about 100 kPa to about 2 MPa, or about 100 kPa to about 7 MPa, or about 100 kPa to about 20 MPa, or even higher. The confined compression strength of the IPN hydrogels may be about 100 kPa to about 1 MPa, or about 100 kPa to about 2 MPa, or about 1 MPa to about 2 MPa. Hydrogels that will remain in a subject's anatomy as temporary or long-term implants may be selected to have a compression strength similar to the relevant natural tissue.

In the devices configured for wound healing, the hydrogel may be configured to absorb wound exudates from a wound environment. However, so that the hydrogel does not adhere to or grow into the wound tissue or surrounding tissues, the hydrogel may be surface treated so as to be nonadhesive to surrounding tissue. For example, hydrogels may be pre-wetted with a predetermined amount of a wetting agent so as to be nonadhesive to surrounding tissue. If the hydrogel is nonadhesive to tissue, then the presence of the hydrogel may not interfere with the closure of tissue, and the hydrogel may be easily removed, repositioned, or replaced with little or no trauma to the healing wound.

Any suitable wetting agent may be used to pre-wet an IPN hydrogel so as to retain its absorptive properties and yet make the IPN hydrogel nonadhesive to tissue. For example, a saline solution such as PBS, water, or glycerol may be used. Wetting agents may include one or more buffering agents to control pH. Any suitable biologically compatible buffering agents may be used, e.g., bicarbonates such as sodium bicarbonate, 4-(2-hydroxyethyl)-1-piperazineethanesulfonic acid (HEPES), carbamoylmethylaminodiacetic acid, 2-[N,N-bis(2-hydroxyethyl)amino]ethanesulfonic acid (BES), N-(2-hydroxyethyl)piperazine-N′(4-butanesulfonic acid) (HEPBS), 3-[4-(2-Hydroxyethyl)-1-piperazinyl]propanesulfonic acid; 4-(2-hydroxyethyl)-1-piperazinepropanesulfonic acid (HEPPS), 3-[[2-hydroxy-1,1-bis(hydroxymethyl)ethyl]amino]-1-propanesulfonic acid (TAPS), and combinations thereof. In some variations, the hydrogel may be pre-wetted with the wetting agent so as to comprise at least about 5%, at least about 10%, at least about 15%, at least about 20%, or at least about 25% by weight of the wetting agent. The amount of wetting agent used in an IPN hydrogel may be predetermined so as to make the hydrogel relatively nonadhesive to tissue, but still sufficiently absorbent to absorb a desired amount of wound exudates or other moisture. Further, the pH, salt concentration and/or buffering agent concentration may be adjusted in a wetting agent to tune its properties. The amount of wetting agent incorporated into a hydrogel may be determined by allowing the hydrogel to soak in a pre-wetting agent for a predetermined amount of time, and/or by weighing the hydrogel.

As described above, the IPN hydrogel in these implants may comprise a first polymer network that comprises a hydrophilic nonionic polymer that is covalently cross-linked between ends, and a second polymer network that has been cross-linked and/or polymerized in the presence of the first network. In some variations, the first polymer network may be formed by cross-linking end-functionalized PEG-based macromonomers. The second polymer network may be formed by cross-linking acrylic acid monomers. In certain variations, an implant comprising an IPN hydrogel may be pre-wetted with a wetting agent, e.g., a saline solution, water, or glycerol, so as to be nonadhesive to surrounding tissue as described above. In other cases, e.g., where the IPN hydrogel is designed to be a long term implant, the IPN hydrogel may adhere to surrounding tissues. In other variations, an IPN hydrogel used in an implant may be adhesive to surrounding tissue.

For application in expandable implants, the properties of the IPN hydrogel may be adjusted accordingly. For example, the expandable implants may be configured to have a predetermined expanded dimension at an equilibrium state, e.g., a cross-sectional dimension and/or a depth dimension, so that once the hydrogel has absorbed a certain quantity of moisture, the dimension of the implant will remain relatively constant. The rate of moisture uptake may be varied to control a rate of tissue expansion. For example, a hydrogel in an implant may be configured to expand progressively in volume over a period of days, weeks, or months (e.g., about 1 to about 4 months) to allow a desired expansion of tissue. In general, a degree of expansion of overlying tissue of up to about 3 times, or about 3.5 times, or about 4 times the original area of that tissue may be desired. In some cases, a hydrogel may be configured to expand in volume about 10% to about 1000% over a period of weeks for the desired tissue expansion. As stated above, the physical properties of a hydrogel as a function of moisture content may be controlled by varying the composition and/or cross-linking density of the first and second polymer networks, e.g., the molecular weight of a PEG-macromonomer used in the first polymer network and/or a degree of cross-linking in the first and/or the second polymer network. For example, the tensile properties, the elastic properties, and/or an equilibrium moisture content of the IPN hydrogel may be varied.

The wound healing devices and implants described herein may have any suitable configuration so as to be used on a subject's body. In general, the devices and implants may be configured to maintain appropriate contact with the wound, while adapting to the shape of the subject's anatomy surrounding the wound. Further, the devices and implants generally have sufficient physical integrity to remain intact as swelling occurs, which may in some cases be dramatic, e.g., up to 1000% in volume or even more. For example, any of the variations of wound healing devices and implants described herein may be configured to be removably secured to skin proximate the wound, e.g., by sutures, adhesives, staples, tissue anchors, clamps, or the like, or combinations thereof. Some devices or implants may comprise IPN hydrogels that are secured to backings or frames or the like. Backings or frames may be flexible or rigid. In some cases, IPN hydrogels may be secured to adhesive backings that may or may not be flexible. Still other variations of IPN hydrogels may be secured to a device or implant housing. For example, an IPN hydrogel used in an implant may be secured to an implant housing or implant carrier. In those variations, the IPN hydrogel portion of the implant may be expandable with moisture uptake but the implant housing or carrier may have a relatively fixed dimension. Other variations of devices or implants may expand preferentially in one dimension over another dimension. For example, hydrogels may be secured along one or two dimensions to a rigid backing or a rigid frame or boundary, but still may expand in a dimension that is not constrained. In some instances, a hydrogel may be secured to a two dimensional surface and substantially constrained in those two dimensions, but may expand in a dimension away from the surface. In certain variations, a hydrogel may be secured in two dimensions to an open frame, but still able to expand in a dimension away from the frame. A hydrogel may be secured to a rod, but still able to expand circumferentially relative to the rod. In still other variations, a hydrogel may be secured to a flexible and/or expandable structure, e.g., a flexible or elastic backing, a flexible or elastic frame, or a flexible or elastic rod, so that the flexible and/or expandable structure can flex and/or at least partially expand as the hydrogel swells. Devices and/or implants may include one or more additional structural elements. For example, a device or implant may comprise a hydrogel surrounding a mesh, web, fibers, and/or the like. The hydrogel may be formed (i.e., polymerized and/or cross-linked) around such a web, mesh or fibers to provide further structural integrity. In some variations, the one or more structural elements may serve one or more additional mechanical functions, e.g., a structural element may be elastic in one or more directions, or may provide tension in one or more directions. In some variations, a structural element may also provide a vulnerary agent, e.g., a mesh can be coated or impregnated with silver to impart antiseptic properties.

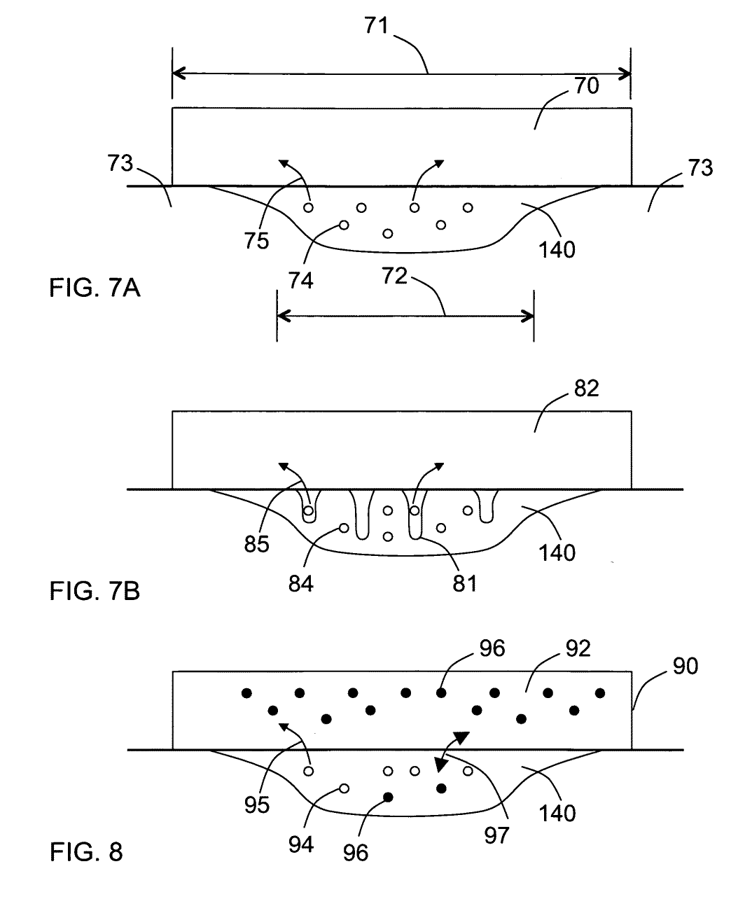

Some devices and/or implants may include one or more wicking members that are configured to absorb moisture. Referring now to FIG. 7A, a wound dressing device 70 is applied over a wound 140. The device 70 has a cross-sectional dimension 71 that is greater than a cross-sectional dimension 72 of the wound 140 so that the device 70 may be supported by dermal tissue 73 surrounding the wound 140. The device 70 absorbs exudates 74 from wound 140, as indicated by arrows 75. Referring now to FIG. 7B, a wound dressing device 80 is applied over a wound 140. This particular device variation has wicking members 81 that extend into the wound 140. The wicking members may draw exudates 84 out of the wound 140 into a body 82 of device 80, as shown by arrows 85. The device body 82 may be configured to absorb high quantities of moisture. If wicking members are present in a device, a density, length, diameter, and/or composition of the wicking members may be varied to adjust a rate and amount of moisture uptake by a device. Although FIG. 7B illustrates a variation of a device with wicking members configured to be used as wound dressings, devices configured to be used as expandable implants may also comprise one or more wicking members, e.g., to allow absorption of moisture from a location that is somewhat remote from the location of the implant.

Some devices and/or implants may comprise an interfacial surface layer designed to interface with tissue, e.g., a wound surface. Such an interfacial layer may in some variations be nonadhesive to surrounding tissue, which may facilitate removal or repositioning of a device or implant, and may prevent or reduce instances where implants or devices become ingrown under tissue. An interfacial layer may in some instances function as a moisture conduit between a device or implant and surrounding tissue, e.g., by wicking moisture away from the surrounding tissue to the device or implant. An interfacial layer may be a contiguous layer, or a noncontiguous layer, and may be uniformly applied to a surface of a device or implant, or preferentially applied to certain portions of a surface of a device or implant. For example, in some variations, an interfacial layer may be primarily applied near the periphery of a device or implant surface, or primarily applied near a central region of a device or implant surface.

The devices and/or implants may have any suitable dimensions, e.g., cross-sectional dimensions and thickness. In general, devices used as wound dressings may have a cross-sectional dimension that is larger than an initial cross-sectional dimension of the wound so that the device can be supported by dermal edges surrounding the wound so that device remains free of the wound during healing, e.g., so that the wound does not heal around an edge or portion of a device. In some variations, the area of a device for a wound dressing may be larger than an area of the wound. A thickness of a hydrogel used as a wound dressing or as an expandable implant may be varied depending on the application, e.g., the amount and rate of moisture that the hydrogel is designed to absorb. In general, the thickness of a hydrogel may be very small, e.g., less than 1 mm, to allow for substantial volume expansion as a function of moisture content. Variations of hydrogels may have an initial (i.e., dry) thickness in the range from about 10 micron to about 20 mm, e.g., about 10 micron, about 20 micron, about 30 micron, about 50 micron, about 80 micron, about 0.1 mm, about 0.2 mm, about 0.3 mm, about 0.5 mm, about 0.8 mm, about 1 mm, about 2 mm, about 5 mm, about 8 mm, about 10 mm, about 15 mm, or about 20 mm.

Some variations of the devices for treating wounds and/or the expandable implants may be configured to deliver one or more vulnerary agents to the subject. Certain variations of the IPN hydrogel devices for treating wounds may be configured to deliver one or more vulnerary agents to the wound as well as to absorb wound exudates, as will be described in further detail below.

A vulnerary agent may be loaded into a hydrogel in any suitable manner, e.g., by swelling, impregnation, and/or infusion. For example, the hydrogel can be formed in the presence of the vulnerary agent so that the vulnerary agent is incorporated into the hydrogel as it forms. In other variations, a hydrogel that has already been formed can be inserted into a solution of the vulnerary agent and allowed to swell with the solution. In the latter case, the amount of vulnerary agent incorporated into the hydrogel may be varied, e.g., by adjusting the concentration of vulnerary agent in the swelling solution, the amount of time that the hydrogel is allowed to swell with the solution, or the temperature at which the swelling occurs.

A hydrogel's properties can be chosen to passively release the vulnerary agent over time, e.g., in a diffusion-controlled manner. In certain variations, a hydrogel may release the agent after initiation, i.e., in response to a stimulus or trigger, e.g., an environmental trigger or stimulus.

Any suitable vulnerary agent that can aid the body in healing tissue and/or promoting desired tissue growth, including vulnerary agents that can prevent infection, and/or reduce scar formation, may be delivered by the devices and implants described herein. Vulnerary agents may include, for example, growth factors, antibodies, antiseptics or antimicrobials such as antibiotics, antifungals, antivirals, anti-parasitic agents, enzymes, buffer agents, vitamins, and combinations thereof. In some variations, vulnerary agents may include therapeutic proteins, or DNA molecules that encode for therapeutic proteins. Vulnerary agents may include an antimicrobial or a combination of antimicrobials that can be applied topically to a wound site. Antimicrobials include antibiotics, antifungal agents, anti-viral agents, anti-parasitic agents, and combinations thereof. Antibiotics may be either bactericidal or bacteriostatic. Examples of antibiotics include aminoglycosides, carbacephems, carbapenems, cephlasporins, glycopeptides, macrolides, monobactam, penicillins, polypeptides, quinolones, sulfonamides, tetracyclines, nitrofurantoin, clindamycin, linezolid, and others, and combinations thereof. Antifungals may include polyene antibiotics, imidazoles, triazoles, allylamines, echinocandin, griseofulvin, flucytosine, fluocinonide, and others, and combinations thereof. Antivirals may include acyclovir, interferons, cidofovir, tromatandine, penciclovir, idoxuridine, vidarabine, trifluridine, and others, and combinations thereof. Antiparasitic agents may include anthelmintics, antihelminthics, antiprotozoal agents, and others, and combinations thereof. In addition, vulnerary agents may comprise a pH-buffering agent or composition, e.g. a buffer solution that can control pH at the wound. Further, vulnerary agents may comprise a compound such as digestive enzyme that can debride the wound, e.g., a protease such as papam.

Vulnerary agents released from the IPN hydrogel may be modified (e.g., encapsulated, coated, and/or complexed) to control or sustain their activity in the wound after they are delivered. For example, vulnerary agents may be encapsulated in a biodegradable polymer carrier that gradually degrades, releasing the vulnerary agent. In other variations, the vulnerary agent may be complexed with a biodegradable carrier, e.g., through electrostatic interaction between a positively-charged growth factor and a negatively-charged carrier. The carrier may degrade to gradually release the vulnerary agent or the carrier may release the vulnerary agent in response to an environmental condition such as pH. Controlled or sustained release may be especially important for growth factors, which can have very short half-lives. By incorporating a growth factor into a carrier, the growth factor may be protected from proteolysis.

Vulnerary agents may be modified such that they are attached to or contained within particles, e.g., particles that have a dimension of about 10 to about 100 nm, or about 100 nm to about 10 μm, or about 10 μm to about 50 μm. For example, vulnerary agents can be at least partially encapsulated with particles of biodegradable molecules, dendrimers or liposomes. In some variations, the vulnerary agents can be incorporated into micelles, e.g., about 20 nm to about 100 nm micelles formed from amphiphilic block copolymers. Biodegradable particles that can be used to incorporate vulnerary agents can be made from many polymers, including poly(lactic acid) (PLA), poly(glycolic acid) (PGA), poly(lactic co-glycolic acid) (PLGA), and polyanhydride.

To heal, treat or manage some wounds, it may be desired to regenerate tissue. Tissue regeneration encompasses regeneration of natural tissues or generation of synthetic substitutes for tissues. Growth factors are often used to aid in tissue regeneration. Injection of growth factors directly on a wound site may have limited efficacy as the growth factor may quickly diffuse away from the desired location. Therefore, in some variations, growth factors may be delivered in a controlled manner to a wound site, e.g., by sustained release or sustained dosing. The type of growth factor used will depend both on the tissue type and the location of the tissue. Growth factors that may be used, alone or in combination, include platelet-derived growth factors (PDGF), epidermal growth factors (EGF), transforming growth factors (TGF) including growth differentiation factor-9 and myostatin (growth differentiation factor-8, GDF-8), acidic fibroblast growth factors (FGF2), basic fibroblast growth factors (FGF1), bone morphogenetic proteins (BMP), hydroxylapatite, hepatocyte growth factors (HGF), vascular endothelial growth factors (VEGF), granulocyte-colony stimulating factors (G-CSF), granulocyte macrophage colony stimulating factors (GM-CSF), neurotrophins including neurotrophin-3 and nerve growth factors (NGF), erythropoietin (EPO), and thrombopoietin. Of course, more than one growth factor may be used in a single device. In particular, some variations of devices may contain two growth factors that have synergistic effects. For example, EGF and VEGF or EGF and PDGF may be combined for synergistic effects. In the latter examples, EGF may increase migration and PDGF may function to enhance fibroblast proliferation. Some examples of the use of combinations of EGF and PDGF are described in Thomas P. Richardson et al., “Polymeric System for Dual Growth Factor Delivery,” Nature Biotechnology 19, 1029-1034 (2001), and Peter Carmeliet and Edward M. Conway, “Growing Better Blood Vessels” (Commentary), Nature Biotechnology 19, 1019-1020 (2001), each of which is hereby incorporated by reference herein in its entirety. In some instances, one or more vulnerary agents to control fibrosis and/or to prevent or reduce scar formation may be used, e.g., anti-TGF-β antibodies may be used.

Any biologic, natural or modified, or peptide-derived segment may be used as a vulnerary agent for the applications described herein. For example, engineered growth factors can induce enhanced biological efficacy and therefore can be selected as a vulnerary agent in the devices described herein. EGF is a 6.2 kDa polypeptide that specifically binds to the epidermal growth factor receptor (EGFR). Binding of EGF to EGFR induces a conformational change and aggregation of its receptors. Receptor aggregation induces tyrosine kinase activity in the cytoplasm of EGFR, which can lead to mitogenic signaling or other cellular activities. Non-limiting examples of engineered EGF mutants are described in Jennifer R. Cochran, et al., “Improved Mutants from Directed Evolution are Biased to Orthologous Substitutions,” Protein Design, Engineering and Selection, 19 (6), 245-253 (2000), and in International Patent Application number WO07/109,673, entitled “Mutant Epidermal Growth Factor Polypeptides, Nucleic Acids, and Uses Therefor,” filed Mar. 20, 2007, each of which is hereby incorporated by reference in its entirety.

The vulnerary agent need not become completely separate from a hydrogel to effectively be applied to a location in a subject's body. Thus, in some variations, a vulnerary agent may be covalently linked to an IPN hydrogel. For example, a vulnerary agent selected from the group consisting of proteins, polypeptides, antibodies, enzymes (e.g., digestive enzymes such as papain), growth factors, amino acids, carbohydrates, lipids, phosphate-containing moieties, hormones, neurotransmitters, nucleic acids, or any combination of biologics, natural or modified, may be chemically linked to an IPN hydrogel. Any suitable method for covalently bonding vulnerary agents to an IPN hydrogel may be used. For example, covalently bonding of a vulnerary agent may include photoinitiated attachment of azidobenzoamido peptides or proteins, photoinitiated functionalization of hydrogels with N-hydroxysuccinimide ester, maleimide, pyridyl disulfide, imodoester, active halogen, carbodiimide, hydrazide, or other functional group followed by reaction, e.g., with peptides or proteins, or chemoselective reaction of aminooxy peptides with carbonyl-containing polymers or monomers. Covalently bonded vulnerary agents may serve a variety of functions, including promotion of epithelial cell adhesion and proliferation on a hydrogel surface. For example, in some variations, the vulnerary agent comprises a growth factor tethered to a delivery hydrogel. Chemical cross-linking agents can be functionalized to allow tethering of the growth factors thereto, and the functionalized chemical cross-linking agents can then be introduced to cross-link and form the delivery hydrogel. The presence of the growth factor in the wound can induce sustained biological signaling through enhanced receptor binding affinity and cell persistence. In some variations, a heterobifunctional cross-linking agent may be used in the formation of the IPN hydrogels that include on azide-active-ester linkages, e.g., 5-azido-2-nitrobenzoyloxy-N-hydroxysuccinimide ester or derivatives thereof, e.g., sulfonated or longer chain derivatives. In some variations, polymeric tethers, e.g., functionalized PEG chains, may be used as intervening moieties between polymer surfaces and covalently attached biologics, and/or between biologics.

As stated above, a hydrogel may be configured to deliver more than one vulnerary agent. For example, a hydrogel may comprise first and second vulnerary agents, or more than two vulnerary agents. The first and second vulnerary agents may be the same or different. A hydrogel may be configured to deliver first and second vulnerary agents concurrently. For example, in some variations, two or more growth factors may be delivered to the wound concurrently. In other variations, a growth factor and another type of vulnerary agent such as an antibiotic may be delivered concurrently to the wound. As will be discussed in more detail below, delivery of multiple, e.g., first and second, vulnerary agents may be initiated separately.

In some variations, an IPN hydrogel may be configured to deliver at least one vulnerary agent in response to an environmental stimulus. The IPN hydrogel's properties may be selected to control delivery of the vulnerary agent, e.g., passive delivery of the vulnerary agent. For example, the IPN hydrogel may be selected or modified to have desired pore size ranges, pore geometries, pore locations, and/or distributions of pore sizes, shapes or locations to control release of the vulnerary agent to the wound. That is, the IPN hydrogel may be chosen or modified to allow certain vulnerary agents to diffuse through its network at a desired rate under the wound's environmental conditions, e.g., temperature and moisture content. In other variations, the IPN hydrogel may be selected to be biodegradable. In these variations, the vulnerary agent may be delivered to the wound as the biodegradable hydrogel degrades over time.

An environmentally-responsive hydrogel may be used in a wound healing device and/or implant, for example, to allow active, .e.g., initiated or triggered, delivery of the vulnerary agent to the wound. Environmentally-responsive hydrogels include three-dimensional cross-linked polymer gel networks that can undergo a volume change in response to an environmental stimulus or trigger, such as temperature, pH, solvent composition, electromagnetic radiation (e.g., light), electric field, ionic concentration (pK), ultrasound energy, specific analytes, or a combination thereof. In some cases, the environmental stimulus may cause a swollen hydrogel to reduce to about 2%, about 3%, about 5%, or about 10% of its swollen volume. Some of these triggers may activate a volumetric change indirectly. That is, the hydrogel may incorporate a material that converts one trigger into another. For example, a molecule that absorbs a certain wavelength of light may be incorporated into the hydrogel. That molecule can then convert light into heat and trigger the hydrogel to undergo a thermally-induced volumetric change.

The volume change of an environmentally-responsive hydrogel may be reversible. For example, the gel network may be able to transition between two equilibrium states, one swollen and one collapsed. The volume change may be discontinuous and rapid, e.g., occurring within seconds. For example, where strong hydrogen bonding interchain interactions form the basis for the cross-linked network, large volume changes can occur over small temperature ranges. Where ionic interchain interactions form the cross-linked network structure, a small change in pH or pK can alter the network to result in a large volume change. In other variations, the volume change may be slower and more continuous. In some applications, hydrogels that can absorb relatively high volumes of wound exudates in a relatively short period of time may be used. In other applications, or in different stages of treatments, hydrogels that absorb wound exudates relatively slowly may be used, e.g., over hours or days. Of course, in certain instances, the same type hydrogel may be used to treat a wound through various stages of wound healing. For example, a hydrogel that has high absorptive capacity may be used throughout a burn treatment, where the full absorptive capacity of the hydrogel may not be used during later stages of healing because of reduced wound exudates.

A variety of environmentally-responsive gels may be used as hydrogels in the wound healing devices and/or implants described herein, allowing the devices and implants to be adapted to a wide range of applications. For example, a thermally-responsive hydrogel may be used to deliver the vulnerary agent at a certain temperature. The thermally-responsive polymer may be pre-swelled with an aqueous solution of a vulnerary agent. As the temperature increases above a phase transition temperature, the polymer network may largely collapse, at least partially expelling solution containing the vulnerary agent into the wound.

A thermally-responsive hydrogel comprising poly(N-isopropylacrylamide) (PNIPAAm) or a derivative, copolymer, or blend thereof can be used. PNIPAAm exhibits a reversible volume phase transition related to a polymer phase separation as the temperature is raised past its lower critical solution temperature (LCST) of about 32° C. A PNIPAAm hydrogel can be loaded with a vulnerary agent by swelling at around 25° C. The loaded PNIPAAm hydrogel collapses at around 37° C., causing the vulnerary agent to be at least partially expelled. Thus, a PNIPAAm hydrogel can be triggered to deliver a vulnerary agent incorporated therein by exposure to human body temperature. For example, a thermally-responsive IPN hydrogel in which the first polymer network comprises PEG, or a copolymer, blend, or derivative thereof (e.g., cross-linked PEG-DA as described above), and the second polymer network may comprise PNIPAAm or a copolymer, blend, or derivative thereof. Here again, the molecular weight of the PEG-based macromonomers (e.g., PEG-DA), the composition of the second polymer network, and/or the cross-linking density may be selected to adjust one or more desired physical properties and/or mechanical properties of the hydrogel, such as absorptive capacity, permeability, brittleness, elastic modulus, flexural modulus, tensile strength, shear strength, shear modulus, stiffness, compressive strength, and other mechanical and physical properties of the hydrogel. For example, PEG-DA macromonomers used to form thermally-responsive PEG-DA/PNIPAAm hydrogels may have a molecular weight at about 1 kDa to about 20 kDa, e.g., about 3.4 kDa to about 8 kDa, about 5 kDa to about 8 kDa, about 6 kDa to about 8 kDa, about 3 kDa to about 6 kDa, or about 4 kDa to about 6 kDa. In PEG-DA/PNIPAAm IPN hydrogels, a molar ratio between the PEG macromonomer and a monomeric component of the second polymer network, e.g., NIPAAm, may be about 1:1, about 1:50, 1:100, about 1:200, about 1:400, about 1:500, about 1:600, about 1:800, or about 1:1000.

In addition to thermally-responsive hydrogels, pH-responsive hydrogels may be used in the wound healing devices and/or implants described herein. Such hydrogels may be tuned to deliver the vulnerary agent when the pH of the wound environment reaches a certain level. Examples of pH-responsive hydrogels include ionic or weakly acidic or basic gels such as PAA, PMAA, and PAAm. PEG can also be modified to contain ionic moieties to form an ionic network that undergoes a volume change in response to a certain pH environment. Some hydrogels configured to deliver a vulnerary agent to a targeted location in a subject's body may also incorporate enzymes or other catalytic entities that may cause the hydrogel to respond to certain biological analytes. For example, the hydrogel may comprise enzymatic cleavage sites in cross-linking molecules that may function to cross-link the network. When the enzymatic cleavage sites are exposed to proteases in a wound site, the hydrogel may collapse, thereby delivering a vulnerary agent to the wound.

Devices may be configured to deliver single or multiple vulnerary agents according to a pre-determined drug-release profile or dosing scheme. For example, a hydrogel may comprise multiple vulnerary agents and deliver them approximately concurrently to the wound. However, at least one of the multiple vulnerary agents may be modified by coating, complexation and/or encapsulation to control its activity after it has been delivered to the wound. For example, a hydrogel may comprise a thermally-responsive delivery hydrogel that incorporates two vulnerary agents, the first unencapsulated, and the second encapsulated by a controlled-release biocompatible polymer. The hydrogel may deliver both vulnerary agents to the wound site at a certain temperature. The first unencapsulated vulnerary agent, e.g., an antibiotic, may have activity relatively quickly, whereas the second vulnerary agent, e.g., another dose of the same antibiotic or a different antibiotic, may have delayed and/or prolonged activity by virtue of its controlled-release coating.

As stated above, some devices, wound dressings, and/or implants may be configured to both absorb moisture (e.g., wound exudates) and to deliver one or more vulnerary agents to a wound. Of course, in some of these devices, a single hydrogel may be configured to perform both functions. For example, referring to FIG. 8, device 90 has body 92 comprising an IPN hydrogel that has been preloaded with a vulnerary agent 96. The device 90 may be preloaded with a vulnerary agent using any scheme or technique described herein or known in the art, e.g., by swelling, by incorporating into a cross-linking agent, and the like. A hydrogel in the device 90 may be configured to release the vulnerary agent 96, e.g., in an active or passive manner as described above, to the wound 140, as indicated by dashed arrow 97. In other variations, the device 90 may also comprise a second hydrogel (which may be the same or different as the hydrogel that releases the vulnerary agent 96) that absorbs wound exudates 94 from wound 140, as indicated by solid arrow 95. Devices comprising multiple hydrogels are described in more detail below. In such device variations, a barrier, e.g., a barrier having low moisture permeability, may be disposed between a relatively wet, swollen hydrogel that is configured to deliver a vulnerary agent and a relatively dry hydrogel that is configured to absorb wound exudates.

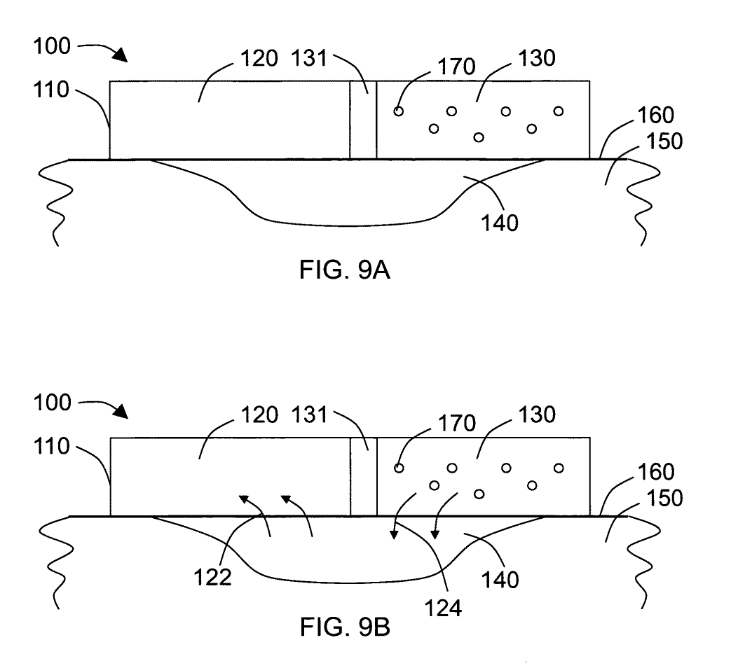

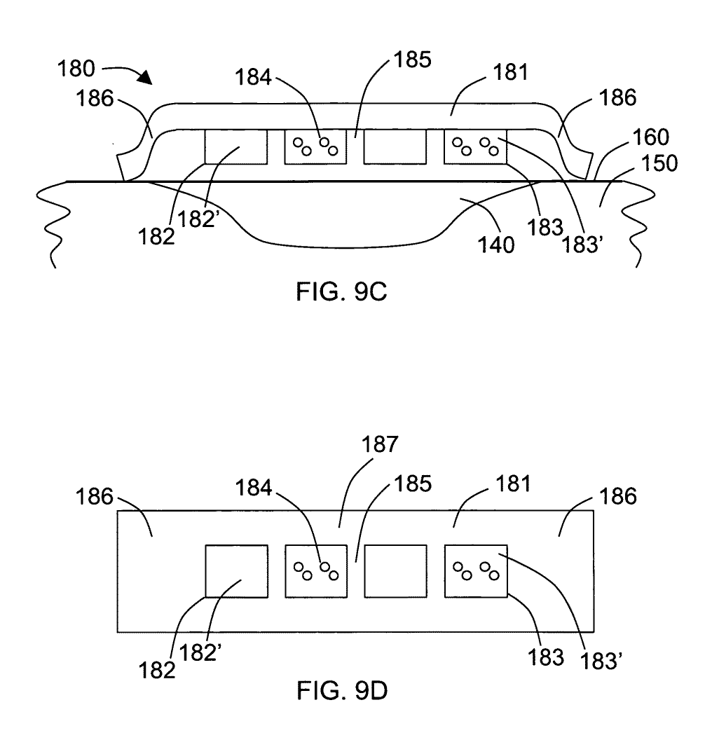

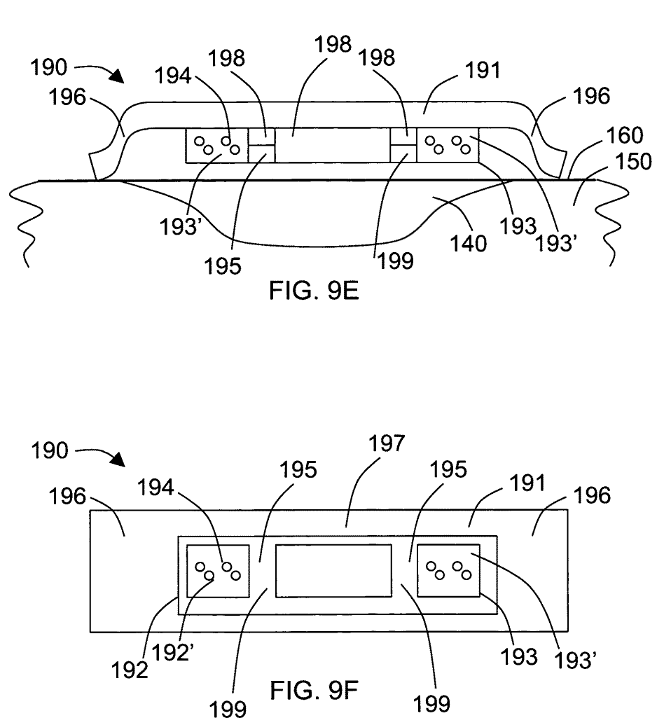

Thus, devices may comprise multiple portions, where one portion may be configured to deliver a vulnerary agent and another portion may be configured to absorb wound exudates. Referring now to FIG. 9A, a device 100 that comprises device body 110 is shown. Device 100 may be removably secured to tissue surrounding wound 140, e.g., to the epidermis 160 and/or to tissue 150 beneath the epidermis. Any suitable mechanism may be used to removably secure device 100 to tissue surrounding the wound, e.g., adhesive, tape, suture, staples, and/or clips. Device body 110 is placed in proximity to wound 140 such that matter can flow between the device body and the wound. For example, at least part of device body 110 may be in continuous or partial contact with wound tissue. In this particular variation, the body 110 comprises a first portion 120 and a second portion 130. Second portion 130 comprises a vulnerary agent 170. As will be discussed in more detail below, vulnerary agent 170 can be incorporated into second portion 130 by any suitable manner, e.g., by mixing, dissolving, impregnating, swelling, bonding, and/or complexing. At least one of the first and second portions of the device body may comprise a hydrogel. As stated above, devices may include one or more additional structural elements. For example, the first absorptive portion and/or the second delivery portion of a device may comprise a hydrogel surrounding a mesh, web, fibers, and/or the like. Such structural elements, if present, may provide one or more additional functions, e.g., they may be elastic in one or more directions, they may provide tension in one or more directions and/or they may provide a scaffold or support for one or more vulnerary agents.

Referring now to FIG. 9B, first portion 120 of a device body 110 is configured to take up or absorb exudates from wound 140, as indicated by solid arrows 122. The first portion of the device body is also referred to as “first absorptive portion” or “absorptive portion” herein. Thus, the first portion 120 can comprise any suitable absorptive material or materials. For example, in some variations, the first portion 120 may comprise a hydrogel capable of absorbing many times its weight in fluid and other debris emanating from a wound. Concentration gradients set up by the absorptive portion may draw unwanted matter away from the wound into the device for disposal. A hydrogel used in the first portion can be referred to as an absorptive hydrogel. Any hydrogel described herein, e.g., any of the IPN hydrogels described above, or known in the art may be used as the absorptive hydrogel.

Referring again to FIG. 9B, the second portion 130 of device body 110 comprises first vulnerary agent 170 and is configured to deliver the first vulnerary agent 170 to the wound 140, as indicated by dashed arrows 124. The second portion of the device body may also be referred to as a “second delivery portion” or “delivery portion” herein. In some variations, second portion 130 can comprise a hydrogel that incorporates the vulnerary agent. The hydrogel may have characteristics that make it capable of delivering the vulnerary agent to the wound. A hydrogel used in the second portion of the device body can be referred to as a delivery hydrogel. Any hydrogel described herein or known in the art may be used as the delivery hydrogel. A delivery hydrogel may be loaded with one or more vulnerary agents by any suitable means, e.g., by swelling, impregnation, and/or infusion, and the delivery hydrogel's properties can be chosen to passively release the vulnerary agent over time, e.g., in a diffusion-controlled manner. In certain variations, the delivery hydrogel can release the vulnerary agent after initiation, i.e., in response to a stimulus or trigger, e.g., an environmental trigger or stimulus, as described above. Of course, a delivery hydrogel may comprise more than one vulnerary agent, and be configured to deliver multiple vulnerary agents concurrently, or separately, e.g., in response to different stimuli.

As is described in more detail below, absorptive portions and delivery portions may have a variety of configurations. For example, an absorptive portion may be physically spaced apart from a delivery portion, or an absorptive portion may be adjacent to a delivery portion. Further, absorptive portions may comprise multiple absorptive subportions, and delivery portions may comprise multiple delivery subportions. Absorptive portions and the delivery portions may have any suitable shape, e.g., they may have rectangular strip-like shapes, or they may be square, round, oval, or custom shaped to fit a certain type of wound or custom-shaped for a specific individual wound. A variety of arrangements of absorptive subportions and delivery subportions are contemplated. For example, some devices may comprise strip-like absorptive subportions interleaved with strip-like delivery subportions, where the strip-like subportions may have similar dimensions or different dimensions.

In some cases, a barrier may be provided between an absorptive hydrogel portion and a delivery hydrogel portion, or between an absorptive hydrogel subportion and a delivery hydrogel subportion. Referring back to FIGS. 9A and 9B, a barrier 131 is positioned between the absorptive hydrogel portion 120 and the delivery hydrogel portion 130. Such a barrier, when present, may be used to prevent or control the transfer of matter, e.g., moisture, from an absorptive hydrogel to a delivery hydrogel. Any suitable barrier material and/or barrier construction may be used. Thus, a barrier may have low water permeability so as to prevent or control moisture transfer from a relatively wet, swollen delivery hydrogel to a relatively dry absorptive hydrogel. Non-limiting examples of suitable low water permeability barriers include polyvinylchloride, polyurethanes, polyolefins such as polyethylene or polypropylenes, latex rubbers such as natural latex rubber, vinyls, and acrylics. In general, a barrier may be flexible so as to accommodate a changing shape and/or volume of a hydrogel and/or contours of a wound or a subject's anatomy. In some variations, a barrier may extend between an absorptive hydrogel and a delivery hydrogel, e.g., a wall-like barrier such as a thin polymer film that has relatively low permeability to a solution used to swell a delivery hydrogel. In certain situations, a barrier may comprise a membrane selected to have certain permeability properties in relation to one or more components or vulnerary agents that may be present a hydrogel, e.g., ionic or molecular species.

In other variations, a barrier may comprise a physical separation or gap between an absorptive hydrogel and a delivery hydrogel. An example of such a device is illustrated in FIGS. 9C-9D. There, device 180 comprises a backing 181, which may be flexible. The backing 181 may comprise end sections 186 that may be secured to tissue, e.g., by adhesive, suturing, stapling, anchoring, and the like. In some variations, the backing 181 may comprise side sections 187 that may also be secured to tissue around the wound 140. In this particular example, absorptive hydrogel 182 comprises multiple subportions 182′ and delivery hydrogel 183 comprises multiple subportions 183′. Absorptive hydrogel subportions 182′ and delivery hydrogel subportions 183′ may be secured to the backing 181, e.g., by using an adhesive, or by using a thread, or the like. The delivery hydrogel subportions 183′ comprise one or more vulnerary agents 184. The absorptive hydrogel subportions 182′ are spaced apart from the delivery hydrogel subportions 183′ by gaps 185. Each intervening gap 185 may itself function as a barrier to limit mass transfer, e.g., moisture transfer, between the absorptive portions and the delivery portions of the device 180. A backing to which absorptive and delivery portions are adhered as illustrated in FIGS. 9C-9D may comprise a hydrophobic, water resistant, or waterproof material such as polyvinylchloride, a polyurethane, latex rubber (e.g., natural latex rubber), a polyolefin, or an acrylic, such that substantial amounts of moisture are not transferred along the backing, e.g., by a wicking action, between absorptive and delivery portions. In some variations, at least some of the gaps, e.g., gaps 185 as shown in FIGS. 9C-9D, between absorptive and delivery hydrogels may be at least partially filled with a hydrophobic, water resistant, or water proof material.

In certain variations of devices, absorptive and delivery portions may be secured to a foundation, or a frame, that may function as a barrier between absorptive portions and delivery portions or between absorptive subportions and delivery subportions. An example of such a device is illustrated in FIGS. 9E-9F. There, device 190 comprises absorption portion 192 and delivery portion 193 that comprises delivery subportions 193′. Although absorptive portion 192 is illustrated in this example as a single strip, other variations may have an absorptive portion that comprises multiple subportions. In this particular variation, the absorptive portion and the delivery portions (including deliver subportions) are secured to a frame or foundation 195 that separates the absorptive hydrogel from the delivery hydrogel and provides a barrier to limit mass transfer (e.g., moisture transfer) therebetween. In some variations, the frame may be flexible. The hydrogels may be secured to the frame in any suitable manner, e.g., using an adhesive, or with a thread or other mechanical attachment. The frame may comprise a material having suitable barrier properties, e.g., low moisture permeability. Non-limiting examples of frame materials include polyvinylchloride, polyurethanes, polyolefins, acrylics, latex rubbers, e.g., natural latex rubbers, and combinations thereof. The frame 195, in turn, may be secured to a backing 191 to secure the device 190 to tissue surrounding the wound 140. For example, lines of adhesive 198 may be applied to the backing 191, and cross-members 199 functioning as barriers between absorptive and delivery hydrogels may be contacted with the adhesive. The backing 191 may be secured to tissue, e.g., via end sections 196 and side sections 197, as described above in connection with FIG. 9D. In this particular variation, the frame 195 extends not only between absorptive and delivery hydrogels, but also around an outer periphery surrounding both absorptive and delivery hydrogels. Of course, variations are contemplated in which a frame extends primarily between absorptive and delivery hydrogels, without extending around an outer periphery.

Absorptive portions and delivery portions of devices may comprise the same hydrogel, or different hydrogels. That is, the absorptive hydrogel and the delivery hydrogel may include some of the same hydrogel components. In some variations, the absorptive hydrogel may be a single polymer network and the delivery hydrogel may be a double polymer network, where one of the polymers in the delivery hydrogel double polymer network is the same as the single polymer in the absorptive hydrogel. For example, the absorptive hydrogel in a device may comprise a PEG hydrogel, and the delivery hydrogel in a device may comprise a PEG hydrogel.

As described above, the second delivery portion of the device body may comprise first and second subportions. The first subportion may comprise a first vulnerary agent and be configured to deliver the first vulnerary agent to the wound. The second subportion may comprise a second vulnerary agent and be configured to deliver the second vulnerary agent to the wound. For example, the first subportion may comprise an environmentally-responsive hydrogel that incorporates the first vulnerary agent and undergoes a volume change in response to a first environmental stimulus to deliver the first vulnerary agent. The second subportion may comprise an environmentally-responsive hydrogel that incorporates the second vulnerary agent and undergoes a volume change in response to a second environmental stimulus to deliver the second vulnerary agent. For example, hydrogels in the first and second subportions can respond to different temperatures, or one hydrogel in one subportion can respond to pH while the other hydrogel in the other subportion can respond to temperature. Such subportions may be separated by a barrier to prevent or control the transfer of matter, e.g., moisture, ions and/or molecular species, therebetween. A barrier, if present, may comprise any suitable material and construction, e.g., a barrier between subportions may comprise a polymer that has low moisture permeability. In some instances, a barrier may comprise a membrane selected to have certain permeability properties in relation to one or more components or vulnerary agents that may be present a hydrogel, e.g., one or more ionic or molecular species.

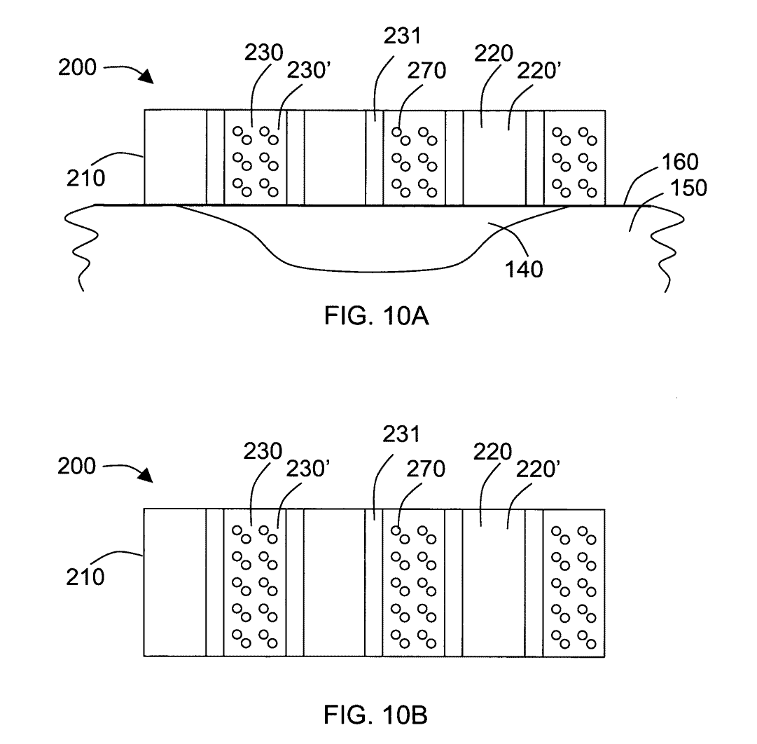

The first and second portions of the device body can be arranged in a variety of suitable geometries. For example, each of the first and second portions can comprise multiple subportions. As illustrated in FIGS. 10A-10B for device 200, the first absorptive portion 220 of body 210 comprises multiple subportions 220′ in the form of bands, and the second delivery portion 230 of body 210 comprises multiple subportions 230′ also in the form of bands. Subportions 230′ comprise vulnerary agent 270. Band subportions 220′ and 230′ can be arranged in the form of alternating bands or stripes. Barriers 231 may be placed between subportions to prevent or control the transfer of matter, e.g., moisture, ions and/or molecular species, therebetween. Although the alternating bands are shown as having approximately equal dimensions for ease of illustration, the bands may have different dimensions. For example, in variations of devices to be used in wounds having excessive wound exudates, the first absorptive portion may have larger total volume than the second delivery portion. Any suitable dimensions can be used for the bands. For example, band dimensions may be adjusted according to wound size or condition. Band dimensions can be adjusted to control diffusion of matter from the first portion to the second portion. For example, bands as narrow as 10 μm or as wide as several mm may be used.

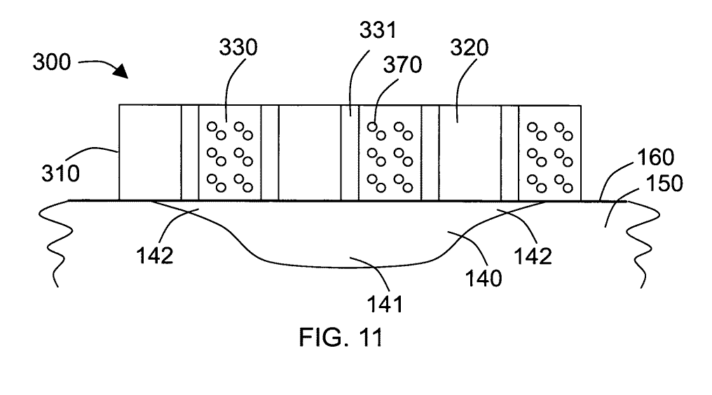

The distribution of subportions making up first and second portions may be varied relative to each other and/or relative to the device body. For example, if the subportions of the first and second portions are arranged in the form of alternating bands, the bands need not be distributed uniformly extending across any dimension of the hydrogel. As illustrated in FIG. 11, a lower band density may be provided near the center of the wound and a higher band density provided near the edges of the wound. For device 300, first portion 320 of body 310 forms alternating bands with second portion 330 comprising vulnerary agent 370. In this variation, barriers 331 may be disposed between adjacent bands to prevent or control the transfer of matter, e.g., moisture, ions and/or molecular species, therebetween. Near the center 141 of wound 140, the spatial density of alternating bands is lower than it is near the peripheral edges 142 of wound 140. In other variations (not shown), the spatial density of alternating bands may be lower near the edges of the wound than near the wound's center.

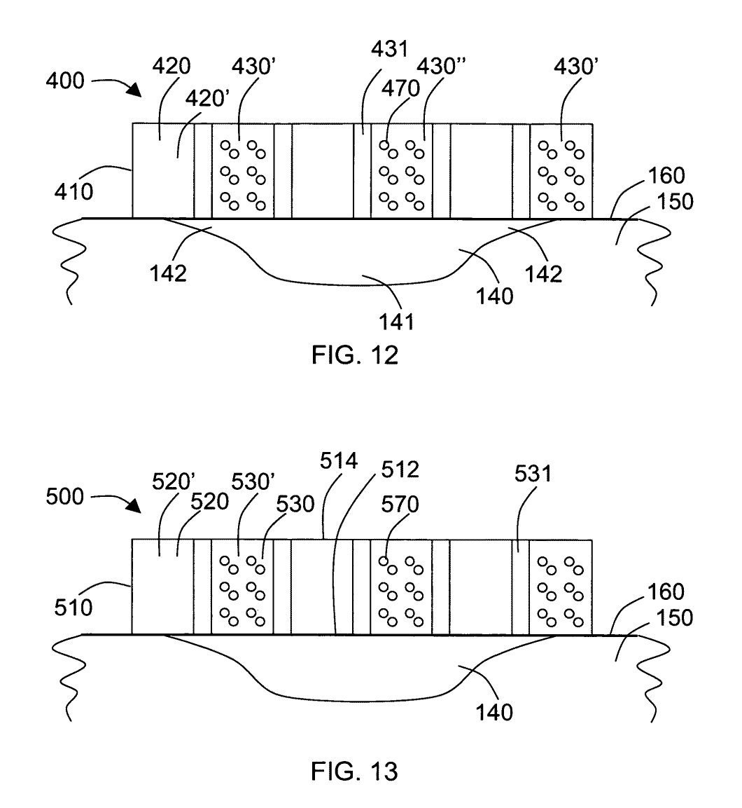

Further, the density or concentration of one or more vulnerary agents can be varied between different delivery subportions in a device. For example, devices can include some delivery subportions having a relatively high vulnerary agent density or concentration and some delivery subportions having a relatively low vulnerary density or concentration. A variation of such a device is illustrated in FIG. 12. Device 400 has device body 410 with first absorptive portion 420 having absorptive subportions 420′ and second delivery portion having delivery subportions 430′, 430″. Barriers 431 may be disposed between subportions to prevent or control the transfer of matter therebetween. In this variation, delivery subportions 430″ contain a higher density of vulnerary agent 470 than delivery subportions 430′. Delivery subportions 430′, 430″ can be in the form of bands alternated with absorptive band subportions 420′. Delivery subportions 430″ containing a higher vulnerary agent density can be positioned near the center 141 of wound 140, whereas delivery subportions 430′ containing a lower vulnerary agent density can be positioned near peripheral edges 142 of wound 140. Of course, delivery subportions having varying densities or concentrations of vulnerary agents can be distributed within devices in any suitable manner.

The density or concentration of one or more vulnerary agents within a delivery portion or subportion may be uniform or nonuniform. Referring now to FIG. 13, device body 510 of device 500 has absorptive portion 520 with absorptive subportions 520′, and delivery portion 530 with delivery subportions 530′. Barriers 531 may be positioned between subportions to prevent or control the transfer of matter therebetween. Vulnerary agent 570 contained within delivery subportions 530′ is nonuniformly distributed within the subportions 530′. In this variation, vulnerary agent 570 is concentrated near the bottom surface 512 of device body 510 adjacent to wound 140. Such a vulnerary agent distribution within a delivery portion or subportion may, for example, allow for increased or tapered rate of delivery of the vulnerary agent to the wound. In other variations (not shown), the vulnerary agent may be concentrated near top surface 514 of device body 510. Such a configuration may, for example, allow for delayed or gradually increasing delivery rate of the vulnerary agent to the wound. In still other variations (also not shown), devices may have combinations of delivery subportions with different vulnerary agent distribution schemes. For example, devices may have any combination of delivery subportions with uniform vulnerary agent distribution, delivery subportions with nonuniform vulnerary agent distribution, delivery subportions with higher vulnerary agent density and delivery subportions with lower vulnerary agent density.

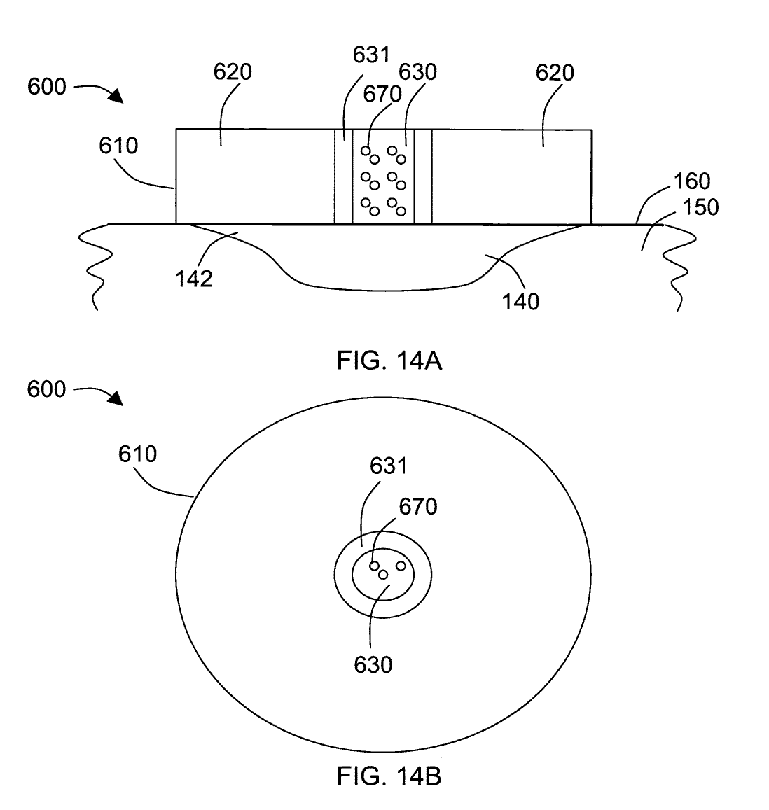

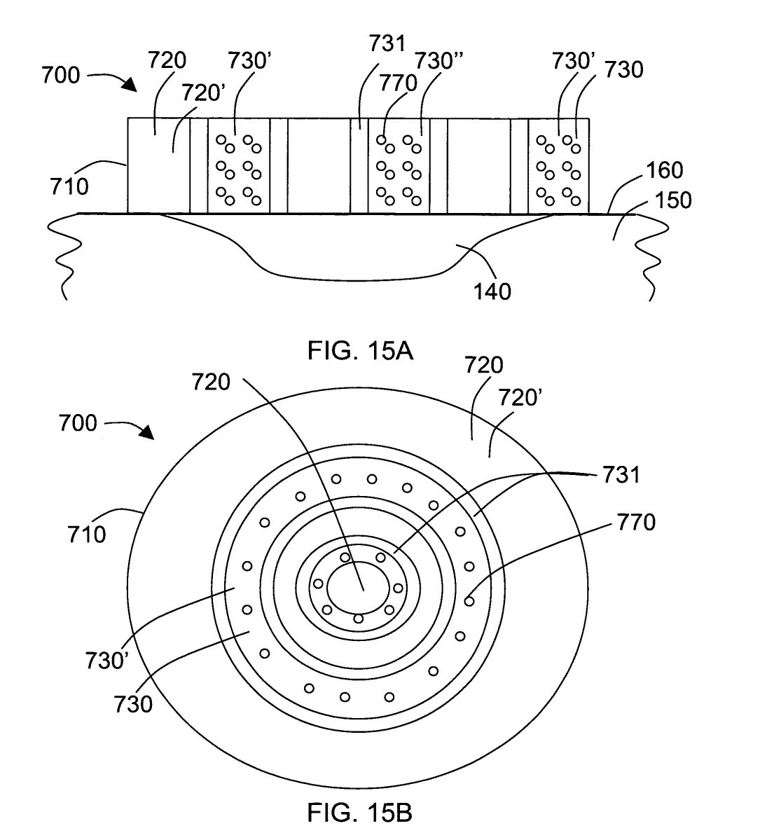

Some variations of devices may comprise first and second portions that take the form of nested, or generally concentric regions. Referring first to FIGS. 14A-14B, device 600 illustrates an exemplary device in which a first portion 620 and second portion 630 of device body 610 are arranged in a generally concentric manner. A barrier 631 may be positioned between the first and second portions to prevent or control the transfer of matter, e.g., moisture, ions and/or molecular species, therebetween. That is, first absorptive portion 620 can form a region, e.g., a concentric region, around second delivery portion 630 comprising vulnerary agent 670. Although not illustrated, it is also contemplated that the second delivery portion may form a region, e.g., a concentric region, around the first absorptive portion. As illustrated in FIGS. 15A-15B, first and/or second portions of the device body can have multiple nested subportions. The multiple nested subportions may be arranged, for example, in a generally concentric configuration. Device 700 includes body 710 which comprises first portion 720 which consists of multiple concentric cylindrical subportions 720′ alternated with multiple concentric cylindrical subportions 730′ of second portion 730. Barriers 731 may be positioned between subportions to prevent or control the transfer of matter therebetween. All subportions 730′ of second portion 730 comprise vulnerary agent 770. Although subportions 720′ and 730′ are depicted as having approximately circular cross-sections, subportions 720′ and 730′ can have any type of cross-sectional shape that can be nested, e.g., in a generally concentric manner. For example, subportions may oval, rectangular, or any type of polyhedral cross-sectional shape.

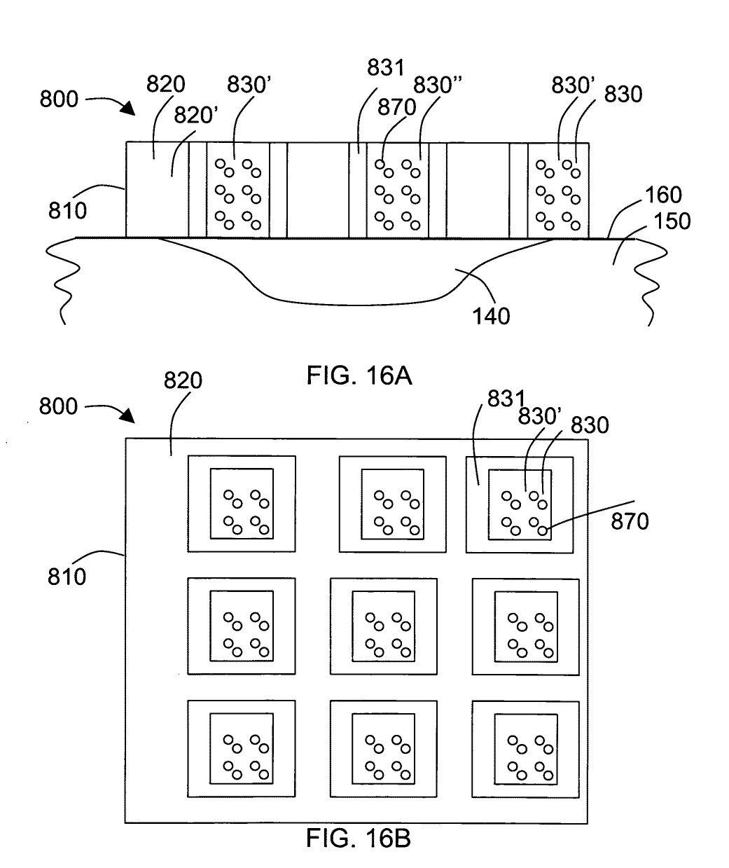

Devices may include first absorptive portions that are contiguous, or have multiple noncontiguous subportions. Some variations of devices include second delivery portions that are contiguous or contain multiple noncontiguous subportions. FIGS. 10A-10B and 15A-15B illustrate devices where subportions of both the first absorptive portion and the second delivery portion are arranged in a noncontiguous manner, whereas FIGS. 9A-9B and FIGS. 14A-14B illustrate devices where both the first and second portions are contiguous. As illustrated in FIGS. 16A-16B, device 800 includes a first absorptive portion 820 of device body 810 that may be contiguous, whereas the second delivery portion 830 of device body 810 may comprise non-contiguous delivery subportions 830′. Barriers 831 may be used to separate the absorptive portion from the delivery portion to prevent or control the transfer of matter therebetween. Each delivery subportion comprises vulnerary agent 870. Although delivery subportions 830′ are illustrated as having an approximately square cross-sectional shapes in FIG. 8, delivery subportions may have any suitable cross-sectional shape, e.g., round, oval, polyhedral, rectangular, etc. Delivery subportions within a single device may have the same or different shapes and/or configurations. Although FIGS. 16A-16B depict delivery subportions 830′ as uniformly distributed throughout first absorptive portion 820, delivery subportions 830′ may be distributed in any suitable manner, e.g., with higher density of delivery subportions near a center region or near an edge region. Of course, variations are contemplated where the first portion comprises non-contiguous absorptive subportions and the second delivery portion is contiguous.

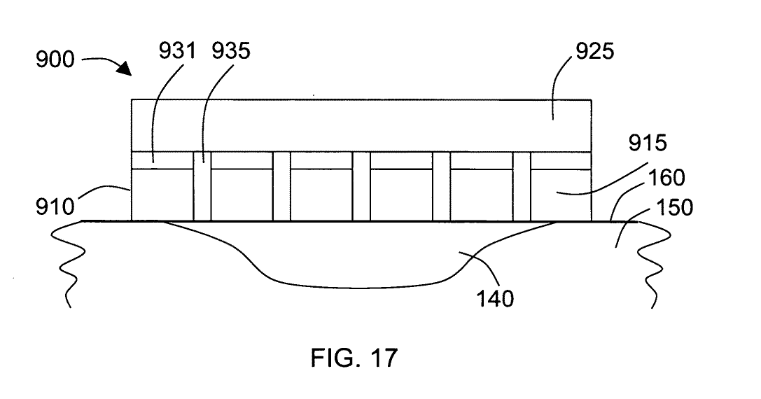

In some variations, the device body may comprise multiple layers. For example, as illustrated in FIG. 17, device 900 comprises body 910 having first layer 915 adjacent to wound 140 and second layer 925 positioned vertically relative to the first layer 915. In some variations, the first layer 915 may comprise the first absorptive portion positioned adjacent to wound 140. The second layer 925 may comprise the second delivery portion including the vulnerary agent. Variations are contemplated wherein the first layer comprises the second delivery portion and the second layer comprises the first absorptive portion. In some variations, matter can transfer between the second layer and the wound by diffusion. In other variations, a barrier layer or partial layer may be placed between the first and second layers to control or prevent transfer of matter, e.g., moisture, ions and/or molecular species therebetween. In some variations, channels 935 may be provided within first layer 915 to allow more direct passage of matter between the second layer and the wound. Channels 935 can be openings or pathways. Channels 935 can also be openings filled with wicking substances or wicks adapted to draw matter out of the second layer and deliver it to the wound or vice versa. In variations including channels, a barrier layer 931 may be used to prevent or control the transfer of matter between layers in regions around the channels.

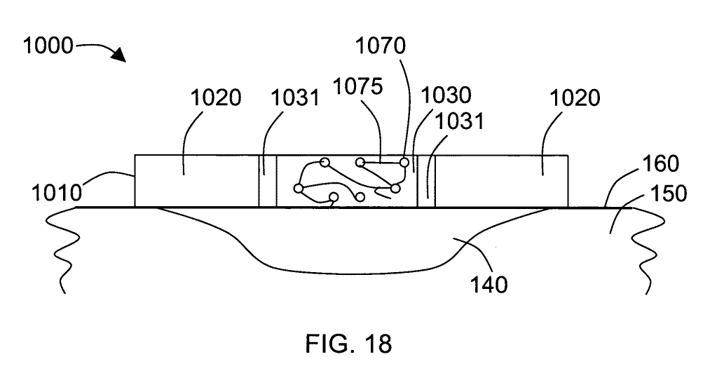

The vulnerary agent need not become completely separated from the second delivery portion of the device to effectively treat a wound. Referring now to FIG. 18, in device 1000 with body 1010 having first absorptive portion 1020 and second delivery portion 1030, the vulnerary agent 1070 is tethered to at least part of the second portion 1030. The first absorptive portion may be separated from the second delivery portion by a barrier 1031. Second portion 1030 can comprise a cross-linked network 1075 to which vulnerary agent 1070 is attached. For example, in some variations, the vulnerary agent comprises a growth factor tethered to a delivery hydrogel. Chemical cross-linking agents can be functionalized to allow tethering of the growth factors thereto, and the functionalized chemical cross-linking agents can then be introduced to cross-link and form the delivery hydrogel. The presence of the growth factor in the wound can induce sustained biological signaling through enhanced receptor binding affinity and cell persistence.

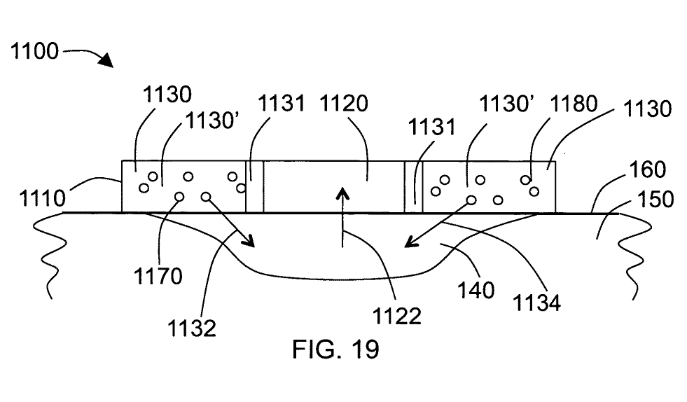

As described above, the delivery portion of the device body may comprise multiple subportions selected to deliver vulnerary agents in response to different stimuli, e.g., environmental stimuli. For example, a delivery portion may comprise a first subportion comprising a first vulnerary agent wherein delivery is initiated by a first stimulus. A delivery portion may also comprise a second subportion comprising a second vulnerary agent wherein delivery is initiated by a second stimulus. For example, as illustrated in FIG. 19, device 1100 comprises body 1110 with first absorptive portion 1120, and delivery portion 1130 comprising first delivery subportion 1130′ and second delivery subportion 1130″. Each delivery subportion may be separated from the absorptive portion by a barrier 1131. First absorptive portion 1120 is configured to absorb wound exudates, as illustrated by solid arrow 1122. Delivery subportion 1130′ is configured to deliver first vulnerary agent 1170 to the wound in response to a first stimulus, as indicated by dashed arrows 1132. Delivery subportion 1130″ is configured to deliver second vulnerary agent 1180 to the wound in respond to a second stimulus, as indicated by dashed arrows 1134. One or both of the first and second stimuli may be an environmental stimulus, e.g., the first and second stimuli may be different temperatures. In other variations, one of the stimuli may be different in nature than the other, e.g., one stimulus may be temperature and the other stimulus may be pH or pK.

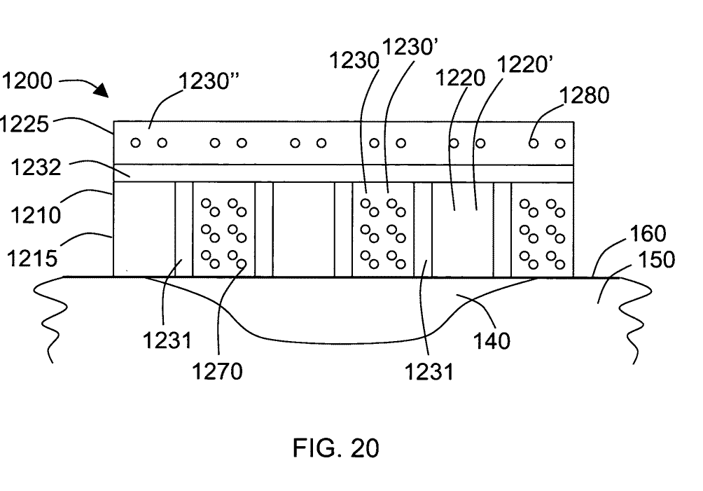

Devices with multiple layers may be provided to deliver more than one vulnerary agent to the wound. For example, as shown in FIG. 20, device 1200 with body 1210 can comprise a first layer 1215 adjacent to wound 140. The first layer 1215 can comprise an absorptive portion 1220, and a delivery portion 1230 comprising a first vulnerary agent 1270. As shown, absorptive portion 1220 can have multiple absorptive subportions 1220′ in first layer 1215 and delivery portion 1230 can have multiple delivery subportions 1230′ in first layer 1215. Delivery subportions may be separated from absorptive subportions by barriers 1231. Body 1210 can comprise a second layer 1225 disposed vertically relative to first layer 1215. Second layer 1225 can comprise another delivery subportion 1230″. A second vulnerary agent 1280 can be provided in delivery subportion 1230″ in second layer 1225. Barrier 1232 may be provided between the first and second layers to prevent or control the transfer of matter therebetween. In some variations, channels, pathways, or wicks (not shown) extending through the first layer can be provided to allow passage of the second vulnerary agent to the wound without requiring diffusion through the first layer. In certain variations, multiple delivery hydrogels disposed in multiple layers can be thermally-responsive hydrogels that undergo a volume change when exposed to body heat. Thus, a delivery hydrogel comprising a first vulnerary agent in a layer adjacent to the wound can be triggered first to deliver the first vulnerary agent, whereas a delivery hydrogel comprising a second vulnerary agent in a second layer isolated from the body via the first layer can deliver the second vulnerary agent in a time-delayed manner. In these variations, the time-delay between delivery of the first and second vulnerary agents can be determined by the heat conduction properties of the first layer (i.e., its thickness and specific heat).

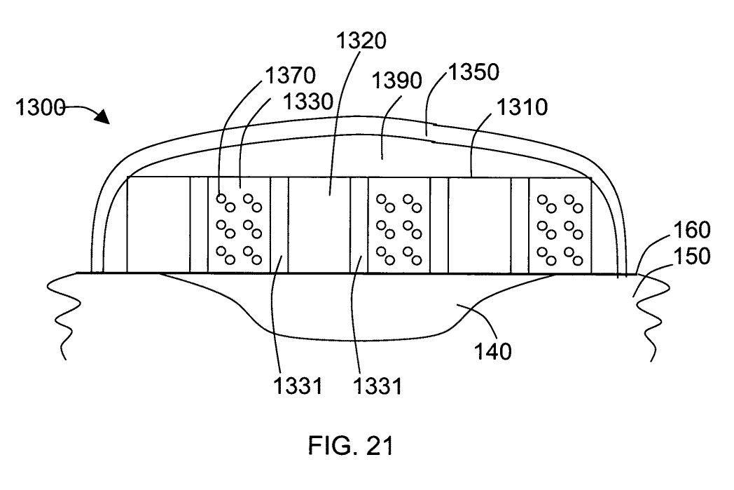

In some variations, the devices can create a negative pressure environment at the wound. In these variations, the negative pressure environment may enhance the devices' ability to extract exudates from the wound through absorptive mechanisms. A “negative pressure” environment at the wound refers to a situation in which the pressure of a fluid surrounding the wound is lower than the pressure within the body. For example, as illustrated in FIG. 21, device 1300 can include a cover 1350 surrounding device body 1310. An airtight seal can be formed between cover 1350 and epidermis 160 to enclose a volume 1390 between wound 140 and cover 1350. Cover 1350 can be made of a material generally impermeable to air, e.g., metal or plastic. Volume 1390 incorporates device body 1310. In some variations, cover 1350 can be rigid, rendering volume 1390 approximately fixed. Cover 1350 can be integral with or separate from device body 1310. First absorptive portion 1320 may be separated from second delivery portion 1330 by barriers 1331. As first portion 1320 absorbs wound exudates out of wound 140 and/or as second portion 1330 changes in volume in response to an environmental condition, the pressure inside volume 1390 can be reduced to below ambient. The amount of pressure change within volume 1390 may depend on a variety of factors, including net volume change of the first and/or second portions of device body 1310, change in vapor pressure from water or other substances present inside volume 1390, and whether or not cover 1350 is rigid. If the pressure inside volume 1390 is reduced below that in the wound, extraction of wound exudates may be accelerated or enhanced. Conversely, if the pressure inside volume 1390 is increased above ambient pressure, e.g., if device 1310 undergoes a net volume increase, that increased pressure may help drive the vulnerary agent into the wound.

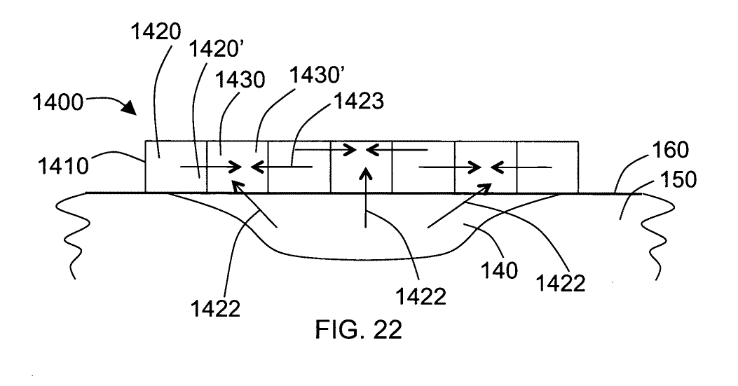

In certain instances, diffusion gradients created by the interaction of the absorptive portions with the delivery portions of the device may enhance absorption of wound exudates. For example, as shown in FIG. 22, absorptive portion 1420 includes multiple subportions 1420′ interspersed between multiple subportions 1430′ of delivery portion 1430. Subportions 1420′ absorb wound exudates as indicated by solid arrows 1422. Wound exudates can then diffuse from subportion 1420′ to subportion 1430′ as indicated by dashed arrows 1423. Diffusion between absorptive portions and delivery portions can be tuned or controlled using barriers, e.g., membranes or walls (not shown) between the absorptive and delivery portions.

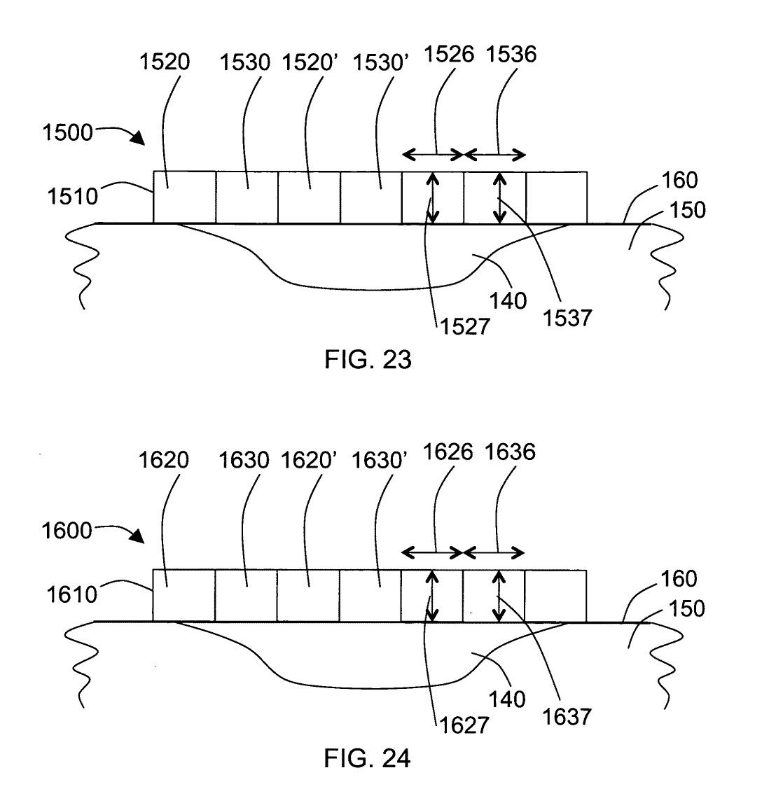

In addition, diffusion driven by concentration gradients can be affected by the configuration and/or geometry of the absorptive and delivery portions. For example, as shown in FIG. 23, device 1500 has body 1510 with absorptive portion 1520 comprising absorptive subportions 1520′ interspersed between delivery subportions 1530′ of delivery portion 1530. Delivery subportions 1530′ have a cross-sectional width 1536 and thickness 1537. Absorptive subportions 1520′ have a cross-sectional width 1526 and thickness 1527. If cross-sectional widths 1526 and 1536 are small relative to thicknesses 1527, 1537, diffusion between absorptive and delivery portions may occur more readily. Devices where diffusion between absorptive and delivery portions is impeded can also be provided. For example, as illustrated in FIG. 24, device 1600 comprises body 1610. Body 1610 includes absorptive portion 1620 comprising absorptive subportions 1620′ and delivery portion 1630 delivery subportions 1630′. Delivery subportions 1630′ have a cross-sectional width 1636 and thickness 1637. Absorptive subportions 1620′ have a cross-sectional width 1626 and thickness 1627. If cross-sectional widths 1626, 1636 are large compared to thicknesses 1627, 1637, diffusion between absorptive and delivery subportion may be relatively slowed or impeded. For the device variations illustrated in FIGS. 23 and 24, one or more barriers (not shown), e.g., membranes, may be used to control diffusion between absorptive and delivery portions.

### II. Methods for Treating Wounds

Any of the hydrogels and devices incorporating hydrogels described above may be used in methods for treating wounds. In general, the methods comprise controlling an amount of moisture in a wound as a function of time. Some methods may comprise applying one or more nonadhesive IPN hydrogels, e.g., one or more pre-wetted IPN hydrogels to a wound. IPN hydrogels may be pre-wetted as described above, e.g., with a saline solution, water, or glycerol, so as to be nonadhesive to the subject's tissue. An IPN hydrogel applied to a wound may comprise a first cross-linked polymer network and a second polymer network that has been polymerized and/or cross-linked in the presence of the first cross-linked network.

Any IPN hydrogel described herein may be applied to the wound. For example, the first polymer network may comprise a hydrophilic, nonionic polymer that is cross-linked between ends, e.g., a first polymer network formed by cross-linking end-functionalized PEG. The second polymer network may comprise a hydrophilic and/or ionizable polymer network, e.g., a PAA polymer network. The amount of moisture in the wound may be controlled as a function of time by adjusting the properties of the first and second polymer networks, and the relative proportions of the first and second polymer networks in the IPN hydrogel. Some variations of the methods may comprise applying a series of hydrogels to a wound. For example, a relatively highly absorptive hydrogel may be applied to a new wound, whereas a hydrogel that absorbs moisture more slowly or reaches an equilibrium content at a lower level may be used as wound healing progresses.

Some variations of the methods for treating wounds may include delivering one or more vulnerary agents to the wound. For example, certain variations of these methods may comprise utilizing a device configured to both absorb wound exudates and to deliver a vulnerary agent, as described above. The delivery of a vulnerary agent may be passive or active, as described above. The methods may include delivering more than one vulnerary agent to a wound. In those cases, multiple vulnerary agents may be selected to act synergistically within the wound. For example, the first vulnerary agent can be a first growth factor and the second vulnerary agent can be a second growth factor. Delivery of a vulnerary agent to the wound can be initiated by an environmental stimulus that causes a volume change in the environmentally-sensitive hydrogel. The environmental stimulus may include temperature, pH, ionic concentration, electromagnetic radiation, electric field, ultrasound energy, or a combination thereof. In some variations of the method, delivery of the vulnerary agent to the wound can be initiated by exposing the device to body temperature.

In the methods for treating wounds, a wide variety of vulnerary agents may be used. For example, the first vulnerary agent can be a growth factor, an antibody, an antifungal, an antibiotic, an antiviral, an anti-parasitic agent, a vitamin, or a combination thereof. Vulnerary agents may include therapeutic proteins, or DNA molecules that encode for therapeutic proteins. Growth factors that may be used in the methods include platelet-derived growth factors, epidermal growth factors, transforming growth factors including growth differentiation factor-9 (GDF-9) and myostatin (growth differentiation factor-8, GDF-8), acidic fibroblast growth factors, basic fibroblast growth factors, bone morphogenetic proteins, hydroxylapatite, hepatocyte growth factors, vascular endothelial growth factors, granulocyte-colony stimulating factors, granulocyte macrophage colony stimulating factors, neurotrophins including neurotrophin-3 and nerve growth factors, erythropoietin, thrombopoietin, and combinations thereof. Any biologic, natural or modified, or peptide-derived fragment may be used as the first vulnerary agent.

The wound treating methods may include devices that are capable of delivering vulnerary agents according to a dosing scheme or staged delivery plan. For example, devices can be configured to deliver the first vulnerary agent to the wound in response to a first stimulus and deliver the second vulnerary agent to the wound in response to a second stimulus. The first and/or second stimulus may be an environmental stimulus, e.g., temperature, pH, ionic concentration, electromagnetic energy, electric field, and/or ultrasound energy.

Some variations of the methods include using devices to create a negative pressure environment around the device body and the wound. The negative pressure environment can assist in the extraction of wound exudates. To form a negative pressure environment, an airtight seal can be formed around the device body and the wound. Such an airtight seal can comprise an air-impermeable cover (e.g., made out of plastic or metal) that is sealed around the periphery of the wound and the device body with an adhesive or tape. Volume changes of hydrogels making up the device body can in some instances cause a net pressure reduction inside the airtight seal. The net pressure reduction may augment the device's ability to extract exudates from the wound.

### IIII. Methods for Expanding Tissue

Any of the hydrogels described above, e.g., IPN hydrogels, may be used in methods for expanding tissue. In general, these methods comprise selecting the first and second polymer networks in an IPN hydrogel to control a dimension of the hydrogel as a function of time to expand the tissue layer, and implanting the IPN hydrogel under a tissue layer and. In some cases, the relative proportions of the first and second polymer networks in the IPN hydrogel may be varied to control a dimension of the hydrogel as a function of time to expand the tissue layer. In certain variations, the methods may comprise delivering one or more vulnerary agents to tissue surrounding the implant, e.g., with the hydrogel in the implant. In some applications, e.g., an IPN hydrogel used to expand tissue may be surface treated, e.g., by pre-wetting, as described above, so as to be relatively nonadhesive to surrounding tissues.

### IV. Kits

Kits comprising the IPN hydrogels described herein are provided. Some variations of the kits may comprise IPN hydrogels for wound treatment, and some variations of the kits may comprise IPN hydrogels for expandable implants.

Kits designed either for wound treatment applications or implant applications may additionally comprise one or more vulnerary agents. The vulnerary agents in the kits may or may not be delivered to the body by a hydrogel in the kit. If present, a vulnerary agent may be any vulnerary agent described herein or known in the art, e.g., a vulnerary agent selected from the group consisting of growth factors, antifungals, antibiotics, antivirals, anti-parasitic agents, vitamins, pH buffering agents or compositions, enzymes (e.g., digestive enzymes such as papain), antibodies, and combinations thereof.

Kits may also comprise one or more fasteners to secure a hydrogel to a body, e.g., a wound site or an implantation site. If present, the fasteners may be any fasteners described herein or known in the art and may comprise for example an adhesive, a suture, a tissue anchor, a staple, a clamp, and combinations thereof. In some variations, kits may comprise one or more tools for visualizing, resizing, securing, positioning, and or removing a hydrogel from a wound. For example, kits may comprise a clamp, a hemostat, a grabber, a hook, a cutter, and the like, and combinations thereof.

Some kits may comprise a pre-sized or pre-shaped hydrogel. In those kits, the hydrogels may be pre-sized for particular applications, e.g., wound types, sizes and/or shapes, or implant types, sizes and/or shapes. Other variations of kits may comprise a generically sized hydrogel, so that a practitioner or user may customize the size and/or shape of the hydrogel for a particular application, wound type, wound size, wound shape, body location, and/or body size. In some variations of kits, one or more patterns may be included to assist or guide a practitioner or user in customizing the size and/or shape of a hydrogel.

Variations of kits designed for wound treatment may comprise one or more IPN hydrogels, one or more wetting agents, and instructions for pre-wetting a hydrogel in the kit with one or more wetting agents with a predetermined amount of the one or more wetting agents so as to make the hydrogel nonadhesive to tissue in and/or around the wound. For example, the kits may comprise instructions for immersing a hydrogel in a wetting agent for a particular period of time so as to take up a predetermined amount of a wetting agent. A kit may also comprise instructions for applying a pre-wetted IPN hydrogel to a wound, e.g., instructions to position the hydrogel in the wound, to secure the hydrogel to the wound, to adjust a moisture content of the hydrogel using an external moisture source, and/or a treatment protocol involving the hydrogel.

Some variations of kits for wound treatment may provide instructions for applying more than one pre-wetted IPN hydrogel to a wound, e.g., in combination and/or in a serial manner. For example, some kits may provide instructions for a treatment protocol that involves the application of one of the hydrogels in the kit to a wound for a first treatment interval that may be defined by a specific period of time, until the wound reaches a particular moisture level, and/or until the wound reaches a particular healing phase, and then applying another of the hydrogels in the kit for second treatment interval, and so on. The kits may also provide instructions for the conjunctive use of more than one hydrogel, e.g., where a more absorbent hydrogel is placed in a more exudative portion of a wound, and a less absorbent hydrogel is placed in a less exudative portion of the wound.

Some kits may comprise a set of implants, where at least one of the set of implants comprises an IPN hydrogel as described herein. In these kits, the IPN hydrogel is configured to be placed under tissue of a subject and to progressively expand the tissue. The set of implants in some kits may contain a series of implants that are designed to placed under the tissue of the subject in a sequential manner to progressively expand the tissue. In certain variations, the set of implants may contain implants that are designed to be used in conjunction with other implants in the set of implants. The other implants in the set may or may not comprise an IPN hydrogel. For example, some kits may comprise one expandable implant comprising an IPN hydrogel, and another type of implant, e.g., a saline-filed implant or other prosthesis.

Described herein are kits for treating wounds, in particular for the treatment or management of chronic wounds. The kits comprise in packaged combination at least first and second devices. The first device is capable of absorbing exudates from the wound and the second device is capable of delivering a vulnerary agent to the wound. The first device can comprise at least one hydrogel, and the second device can comprise at least one hydrogel. For example, the first device can comprise a hydrophilic hydrogel and the second device can comprise an environmentally-sensitive hydrogel that undergoes a volume change in response to an environmental trigger or stimulus. In those variations, the second device can be capable of delivering a vulnerary agent to the wound after initiation by that environmental trigger or stimulus. In other variations of the kits, the second device can be capable of delivering at least two vulnerary agents to the wound. Some kits can comprise a heat source, an ultrasound source, a solution for adjusting pH, a solution for adjusting ionic concentration, an electric field source, an electromagnetic radiation source, or a combination thereof to initiate delivery of the vulnerary agent from the second device to the wound. Kits can also include instructions for use.

Additional kits for treating wounds are described. These kits comprise in packaged combination at least two devices, wherein each device is capable of absorbing exudates from the wound and delivering a vulnerary agent to the wound. In some variations of the kits, the delivery of the vulnerary agent can be initiated, e.g., by a trigger or stimulus, in at least one of the at least two devices. For example, one of the devices can comprise an environmentally-sensitive hydrogel that undergoes a volume change in response to an environmental stimulus or trigger. A hydrogel that undergoes a volume change in response to temperature, pH, ionic concentration, electromagnetic radiation, electric field, ultrasound energy, or a combination thereof may be used. Thus, these kits may comprise a heat source, an ultrasound source, a solution for adjusting pH, a solution for adjusting ionic concentration, an electric field source, an electromagnetic radiation source, or a combination thereof, to initiate delivery of the vulnerary agent to the wound. Kits may also include instructions for use.

### V. Methods for Making Devices Comprising a Delivery Portion and an Absorptive Portion

In addition to the methods described above for making devices and implants described above, e.g., in connection with FIGS. 1, 2A-2C, 3A-3D, and 4A-4D, other methods for making a wound healing devices are described. The methods comprise irradiating a precursor composition containing a hydrogel precursor to form a hydrogel that defines at least one of the first and second portions of the device body. The hydrogel precursor may have any suitable form, e.g., the hydrogel may comprise monomers, macromonomers, oligomers, cross-linking agents or mixtures or solutions thereof. In addition, the hydrogel precursor may comprise more than one type of monomer, macromonomer, or oligomer in a predetermined proportion to allow formation of desired copolymers. The hydrogel precursor may comprise one or more photoreactive components that can interact with an incident light source to cause the one or more photoreactive components in the hydrogel precursor to polymerize and/or cross-link. In some variations, a photoreactive component can be included in the precursor composition as a component separate from the hydrogel precursor, e.g., the precursor composition can be a solution of a hydrogel precursor with a photoreactive component. In some variations, the radiation source may be a light beam having a wavelength in the range from about 250 nm to about 450 nm and the photoreactive component may be reactive to light in that wavelength range. In some variations, the hydrogel precursor may include a free radical initiator, such as a UV-sensitive free radical initiator.

The incident radiation may form a pattern on the hydrogel precursor. Thus, cross-linking and/or polymerization of the hydrogel precursor induced by the incident radiation can reflect the pattern of incident radiation. The resulting spatially-varying pattern of photoinduced cross-linking or polymerization can define the first and second portions of the device body.

Incident radiation that has a spatially-varying intensity pattern can be provided by any suitable technique, e.g., by passing the light source through a mask, filter or grating, or by reflecting the light from a patterned reflective surface. In addition, two or more beams of coherent radiation can be caused to interfere, and the resulting interference pattern can be incident upon the hydrogel precursor to cause a spatially-varying pattern of polymerization and/or cross-linking.

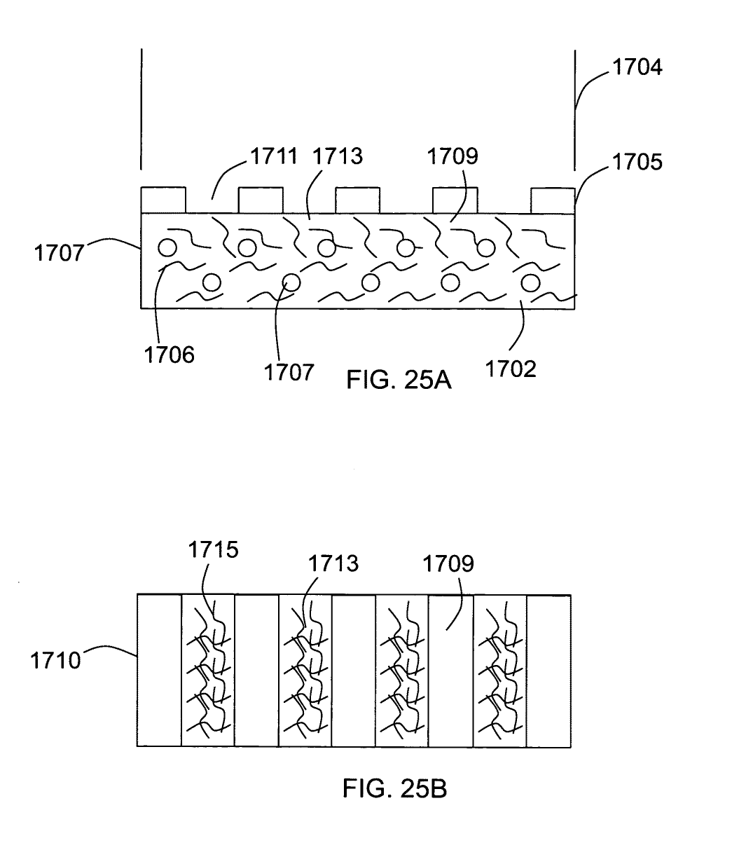

Referring now to FIG. 25A, a precursor composition 1702 comprising hydrogel precursor 1706 and photoreactive component 1707 is irradiated with light beam 1704 passing through mask 1705 having apertures 1711. A photoreactive component can be a part of (e.g., covalently bonded to) a constituent of a precursor composition (e.g., part of a hydrogel precursor), or a photoreactive component can be a separate constituent of a precursor composition. Precursor compositions may contain more than one type of photoreactive component. Hydrogel precursors can be any suitable compound, mixture of compounds, or solution of compounds, e.g., monomers, oligomers, macromonomers, cross-linking agents, and combinations thereof. As shown in FIGS. 25A and 25B, light passing through apertures 1711 of mask 1705 can cause formation of a first cross-linked hydrogel network 1715 in irradiated regions 1713 without substantially affecting nonirradiated regions 1709. For purposes of illustration, apertures 1711 are depicted as parallel slits in mask 1705 to form a pattern of alternating bands as shown in FIG. 22B. However, apertures 1711 can be arranged in any suitable pattern. In some variations, the nonirradiated regions will form the first absorptive portion of the wound-healing devices, and the radiated regions will form a matrix for the second delivery portion of the device body. In other variations, the nonirradiated regions will form the matrix of the delivery portion and the irradiated portion will form the absorptive portion. After the cross-linked hydrogel network is formed in irradiated regions 1713, nonirradiated regions 1709 can be further processed to form a second cross-linked hydrogel network therein. For example, device body 1710 can be heated to cause thermal cross-linking or polymerization in non-irradiated regions 1709. Additional components such as monomers, photoinitiators, or cross-linking agents can be incorporated into nonirradiated regions 1709 before further processing. Device body 1710 can also be irradiated a second time with mask 1705 removed to allow cross-linking or polymerization in regions 1709. Device body 1710 can undergo final processing such as washing to remove unreacted components or impurities, drying with heat, vacuum and/or exposure to desiccant, heating, or further irradiation. As a result of the different processing conditions and/or different compositions of regions 1709 and 1713, these segregated portions of the device body can be designed to comprise distinct hydrogel networks with distinct properties: one having absorptive properties and one having capability to deliver a vulnerary agent. Thus the combined regions 1709 can form the first absorptive portion of the device body while the combined regions 1713 can form the matrix for second delivery portion. Alternatively, the combined regions 1713 can be the first absorptive portion of the device body and the combined regions 1709 can be the matrix for the second delivery portion of the device body.

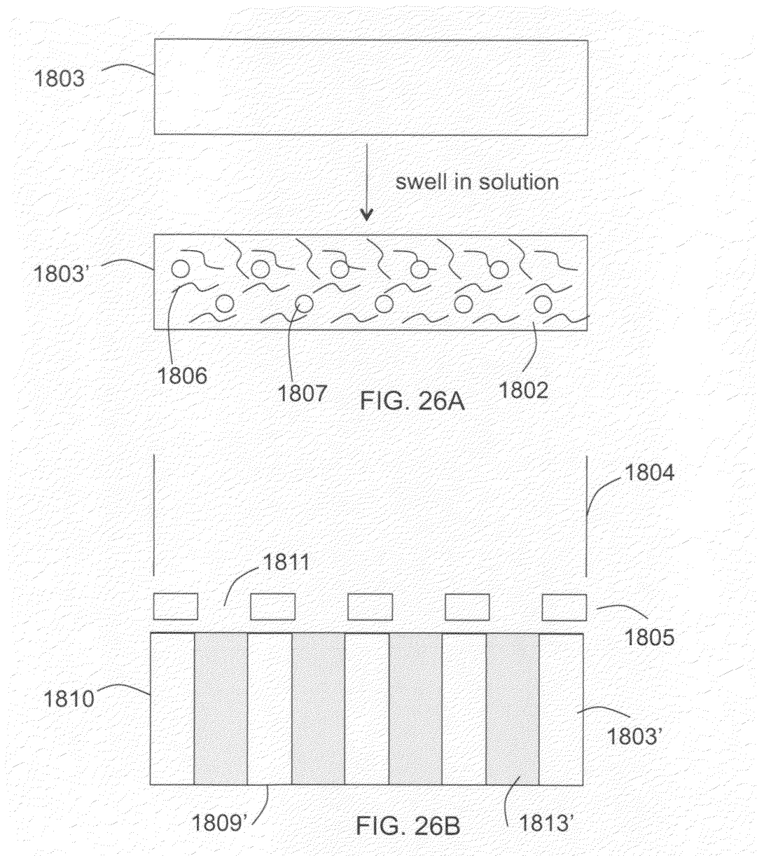

In some variations, the methods for making wound-healing devices can include irradiating a matrix hydrogel that is infused, swollen, or impregnated with an aqueous solution of hydrogel precursor. Referring now to FIG. 26A, matrix hydrogel 1803 is swollen with a composition 1802 (e.g., an aqueous solution) comprising hydrogel precursor 1806 and photoreactive component 1807 to form swollen matrix hydrogel 1803′. Photoreactive component 1807 can be part of (e.g., covalently bonded to) hydrogel precursor 1806, or photoreactive component 1807 can be a separate component of composition 1802. Hydrogel precursor 1806 can be any suitable compound, mixture of compounds, or solution of compounds, e.g., monomers, oligomers, macromonomers, cross-linking agents, and combinations thereof. Then, swollen matrix hydrogel 1803′ can be irradiated with light having a spatially varying intensity pattern as illustrated in FIG. 26B. For example, incident light beam 1804 can be passed through mask 1805 having apertures 1811 to provide irradiated regions 1813′ and nonirradiated regions 1809′ in swollen hydrogel matrix 1803′. Thus, the irradiated regions 1813′ comprise a double interpenetrating network formed between the matrix hydrogel 1803 and the cross-linked hydrogel formed from hydrogel precursor 1806. The nonirradiated regions 1809′ comprise the matrix hydrogel 1803. Device body 1810 can then be washed to remove unreacted components or impurities. Thereafter, device body 1810 can be dried in the presence of heat and/or vacuum. The properties of nonirradiated hydrogel regions 1809′ can be selected to form the absorptive portion of the device body or the delivery portion of the device body. The properties of double hydrogel network irradiated regions 1813′ can be selected to form the other of the absorptive or delivery portion. As described above, barriers may be provided between absorptive and delivery portions. Barriers may be provided before or after any of the cross-linking steps described herein. If provided before patterned cross-linking using irradiation through a mask, the mask may be aligned with the existing barriers. In some variations, a barrier may be formed by forming a gap between absorptive and delivery portions, and then at least partially backfilling the gap with a barrier material. In some variations of the methods, the double network regions 1813′ can comprise a double hydrogel network that undergoes a volume change in response to an environmental stimulus, is loaded with a vulnerary agent and delivers the vulnerary agent to the wound after exposure to the environmental stimulus. In some variations of the methods, vulnerary agent 1807 may be loaded into double network regions 1813′ by swelling matrix hydrogel 1803 with an aqueous solution of vulnerary agent 1807 before irradiation. In other variations of the methods, vulnerary agent 1807 may be introduced as part of hydrogel precursor 1804, e.g., the vulnerary agent may be covalently bonded to a chemical cross-linking agent.

### Example 1

For Example 1, various PEG-DA macromonomers having molecular weights ranging from about 1.5 kDa to about 20 kDa were prepared by reacting various PEG polymers having a molecular weight of about 1.5 kDa to about 20 kDA with acryloyl chloride in tetrahydrofuran (THF) for approximately five hours. Each PEG polymer was dried from toluene, and re-dissolved in tetrahydrofuran (550 ml per 100 g) and kept under nitrogen. Distilled triethylamine (2.5 equivalent per OH group) was added slowly. Then acryloyl chloride was added via dropping funnel (diluted with tetrahydrofuran) over 30 minutes at room temperature. The reaction was allowed to proceed overnight. Filtration was carried out to remove the formed salt by extraction, or using a cellulose membrane ultrafiltration apparatus. For extraction, the volume of the solvent was reduced using a rotary evaporator, and precipitation of the solids was carried out in diethyl ether. After precipitation from diethylether, the raw product was dissolved in methanol and dried in a rotary evaporator. The product was then dissolved in water and filtered through a membrane, and freeze dried.

Each PEG-DA macromonomer was dissolved in water at about 50 wt. %, along with a photoinitiator (2-hydroxy-2-methyl propiophenone) at about 1 vol. % relative to the PEG-DA. The solutions of the PEG-DA macromonomers and the photoinitiator were each placed in a transparent mold and irradiated with a UV source (365 nm, 30 mW/cm²) for 5 minutes. To incorporate the second network, the hydrogel was removed from the mold and immersed at about 50 vol. % in acrylic acid solution with about 1 vol. % (relative to the monomer) 2-hydroxy-2-methyl propiophenone photoinitiator, and about 1 vol. % (relative to the monomer) triethylene glycol dimethacrylate as a cross-linking agent for about 24 hours at room temperature. The swollen gel was then placed between glass plates spaced apart by Teflon spacers and exposed to the UV source (365 nm, 30 mW/cm²) for 5 minutes. In this way, the acrylic acid monomer was polymerized within the PEG network to form an interpenetrating polymer network structure. Following synthesis, the PEG/PAA hydrogels were washed extensively in phosphate buffered saline (PBS) with repeated solvent exchanges for 5 days to remove any unreacted components and to facilitate equilibrium swelling. The PEG/PAA IPN hydrogels were then dried.

Two separate 1 cm diameter excisional wounds were created in each back of mouse models. A circular silicone stent was sewn in place around each wound to prevent excessive healing due to contracture rather than from re-epithelialization, e.g., to simulate a degree of contracture healing of the excisional wound similar to that in humans. Excisional wounds in humans heal approximately 20% by contracture, whereas those in mice heal approximately 80% by contracture. One hydrogel disc having the same diameter as the wound (about 1 cm) was placed in each mouse wound and allowed to remain on the wound for 2 days.

After 2 days, the hydrogel discs were removed from the wounds and a fresh hydrogel disc was applied to each wound. The hydrogels absorbed the most moisture during the first two-day period following creation of the wound. Further, the hydrogels made according to Example 1 using a higher molecular weight PEG-DA macromonomer absorbed more moisture over a two day period than analogous hydrogels made according to Example 1.

### Example 2

Various PEG-diacrylamide macromonomers having molecular weights in the range from about 1.5 kDa to about 20 kDa will be made by azeotropically distilling 100 g of a PEG polymer having a molecular weight of about 1.5 kDa to about 20 kDA in 700 ml toluene under nitrogen, and then removing about 300 ml of toluene. The toluene will then be completely evaporated and the PEG re-dissolved in anhydrous tetrahydrofuran. The solution will be cooled in a room temperature bath under nitrogen and then cooled in an ice bath. Anhydrous dichloromethane will be added until the solution becomes clear (about 100 ml). About 24.6 ml triethylamine that has been distilled prior to use will be added dropwise with stirring. About 13.65 ml mesyl chloride (for an excess of about 3 molar equivalents per OH end group on the PEG polymer) will be added dropwise to the solution. The reaction will be allowed to proceed overnight under argon. The resulting solution will then be vacuum filtered through paper until clear, and subsequently precipitated in diethyl ether. The resulting PEG-dimesylate product will then be added to 400 ml 25% aqueous ammonia solution into a 1 liter bottle, sealed with a Parafilm cover, and vigorously stirred for 4 days at room temperature. Subsequently, the cover will be removed and ammonia will be allowed to evaporate for 3 days. The pH of the solution will be raised to 13 with 1 N NaOH. The solution will be extracted with 100 ml dichloromethane by adding about 5 g NaCl to the water phase. Several more extractions of the water phase with 150 ml aliquots of dichloromethane will be completed. The dichloromethane washes will be combined, concentrated under vacuum, precipitated in diethyl ether, and dried under vacuum. The resulting PEG-diamine will then be azeotropically distilled in 400 ml of toluene under nitrogen, removing about 100 ml toluene. The toluene will then be completely evaporated and the resulting solid re-dissolved in anhydrous tetrahydrofuran. The solution will be cooled to room temperature under nitrogen and then cooled in an ice bath. About 2.46 ml triethylamine (distilled before use) will be added dropwise with stirring, followed by dropwise addition of about 1.43 ml of acryloyl chloride. The reaction will be allowed to proceed overnight under nitrogen. The solution will then be vacuum filtered through paper until clear, followed by precipitation in diethyl ether. The product will be collected by filtration, dried under vacuum, and dissolved in 200 ml deionized water with about 10 g NaCl. The pH of the solution will be adjusted to about 6 with 1 N NaOH, and the solution will be extracted three times with 100 ml dichloromethane, with some product remaining in the water phase as an emulsion. The dichloromethane washes will be combined and precipitated with diethyl ether, after which the solid will be dried under vacuum, or re-dissolved in methanol and then purified by centrifugal ultrafiltration in water through a cellulose membrane having a suitable molecular weight cutoff. The product will be freeze dried.

Each of the resulting PEG-diacrylamide macromonomers will then be cross-linked as described in Example 1 to form a first polymer network. A second cross-linked PAA network will be interpenetrated with each first polymer network as described for Example 1. The resulting interpenetrating network hydrogels will be used as a wound dressing. Moisture uptake properties of the hydrogels will be measured, e.g., as a function of PEG-diacrylamide macromonomer molecular weight used to form the first cross-linked network.

### Example 3

Difunctional allyl ether PEG-based macromonomers will be synthesized from PEG polymers using the following procedure. About 100 ml fresh anhydrous tetrahydrofuran will be added to about 10 g of PEG polymer having a molecular weight in the range from about 1.5 kDa to about 20 kDa (e.g., available from Aldrich). This mixture will be gently heated until the PEG polymer dissolves. The solution will be cooled with an ice bath. About 1.05 molar equivalents of NaH will be added for each PEG reactive OH group. After the release of hydrogen, the reaction container will be purged with argon and allyl chloride or allyl bromide, using 1.1 molar equivalent per PEG OH group, diluted 1:10 in tetrahydrofuran, will be added dropwise using an addition funnel. The reaction mixture will then be heated using an 85° C. oil bath and refluxed overnight. Vacuum filtration will be used to remove sodium bromide by product, and rotary evaporation will be used to reduce the amount of tetrahydrofuran solvent. The resulting PEG-allyl ether macromonomers will be precipitated from solution using iced diethyl ether, with a 10:1 diethyl ether:THF solution.

Each of the resulting PEG-allyl ether macromonomers will then be cross-linked as described in Example 1 to form a first polymer network. A second cross-linked PAA network will be interpenetrated with each first polymer network as described for Example 1. The resulting interpenetrating network hydrogels will be used as a wound dressing. Moisture uptake properties of the hydrogels will be measured, e.g., as a function of PEG-allyl ether macromonomer molecular weight used to form the first cross-linked network.

### Example 4

Hydrogels with interpenetrating networks comprising PEG and PNIPAAm will be used to form the delivery portion of the device body of a device to deliver a vulnerary agent to the wound.

First, a PEG hydrogel network will be synthesized as follows. Poly(ethylene glycol) diacrylate (PEG-DA) will be synthesized by reacting PEG (M.W. about 8000 Da) with acryloyl chloride in tetrahydrofuran for approximately five hours, as described for Example 1. The PEG-DA and 2-hydroxy 2-methyl propiophenone (about 1 vol. % relative to the PEG-DA) will be dissolved in water. The aqueous solution of PEG-DA and the photoinitiator will then be placed in a transparent container made from two 1″×2″ glass slides separated by a 1000 μm spacer. The transparent container will then be exposed to about one 4 square inch UV light in the wavelength range from about 200 nm to about 400 nm from a Xenon lamp for about 10 minutes to photopolymerize the PEG-DA to form a PEG hydrogel network.

Second, a PNIPAAm polymer network will be synthesized within the PEG network to interpenetrate the PEG network. The PEG hydrogel formed above will be swelled with a polymer precursor form of PNIPAAm, which will be photopolymerized in situ. The PEG hydrogel formed above will be removed from its transparent container to provide a gel sample of approximately 1 mm thickness. The PEG hydrogel sample will be immersed in and swelled with an aqueous solution of PNIPAAm monomer, N,N′-methylene bis(acrylamide) (a cross-linking agent) and 2-hydroxy 2-methyl propiophenone at room temperature. The swelled PEG hydrogel sample will subsequently be cooled for about 15 minutes at about 5° C. The swelled PEG hydrogel sample will then be removed from solution and purged in nitrogen gas for about 10 minutes. The swelled PEG hydrogel sample swelled with the PNIPAAm polymer precursor will again be photopolymerized by exposing to a 4 square inch UV light (wavelength in the range from about 200 nm to about 400 nm from a Xenon lamp) for about 10 minutes. After the second photopolymerization, the resulting PEG/NIPAAm IPN hydrogel will be washed for about 48 hours in deionized water to remove any residual photoinitiator or monomer and dried in a desiccator for at least about 12 hours.

Fluorescently labeled wild-type EGF will be incorporated into the water-swollen PEG/PNPIAAm IPN hydrogel by spreading the fluorescently-labeled EGF over the cross-linked hydrogel and allowing reaction to proceed overnight at 25° C. in the dark. The resulting hydrogel will be subsequently placed in about 20 ml of phosphate buffered saline (PBS). Alexa Fluor 488, a fluorescent species, will be covalently bound to the wild-type EGF. A first subset of the samples will then be heated to about 37° C. A second subset of the samples will be held at about 25° C. or lower to maintain the temperature of the hydrogel below its LCST. The PBS solutions for both the first and second subsets will be regularly collected as aliquots and replaced with 2 ml of fresh PBS. The amount of fluorescent EGF dispelled from the hydrogel and into the PBS solution will be measured over time using fluorescence spectroscopy of the collected aliquots.

The release of the EGF from IPN hydrogels into PBS solution over time will be quantified using spectroscopy to model the delivery of the vulnerary agent to the wound in response to body temperature. The intensity of fluorescence emission from the covalently-bound Alexa Fluor 488 at 514 nm following excitation at 488 nm can be correlated with the concentration of the fluorescently-tagged EGF in solution.

Bioassays will be used to determine the effectiveness of the wild-type EGF released from the hydrogel to stimulate a biological response. Immortalized human fibroblasts (BJ-5ta; ATCC; 2×10⁴) will be seeded into 96-well tissue culture plates and conditioned for about 24 h in a serum-free medium. PEG/PNIPAAm hydrogels incorporating the wild-type EGF will be applied to fibroblast monolayers for 24, 48 and 72 h at about 37° C.

Cell survival assays will be performed using the following procedure. At each time point, the PEG/PNIPAAm hydrogels will be removed from the cell monolayers and the cell monolayers will be incubated with Calcein-AM (1 μM)/Ethidum-D (4 μM) (Live/Dead Viability/Cytotoxicity Kit, available from Molecular Probes, Inc.) at 37° C. for about 30 min. Live and dead cells will be quantified by measuring fluorescence intensity using a microplate reader (Gemini EM, available from Molecular Devices Corp.).

Cell proliferation assays will be performed using the following procedure. At each time point (24, 48 and 72 h), the PEG/PNPIAAm hydrogels will be removed from the cell monolayers and cell proliferation will be measured, e.g., by adding WST-8 salt (available from Dojindo Laboratories, Inc.) to the cell monolayer for about 2 h or by adding the alamarBlue assay (reagent available from Invitrogen, Inc.). Cell proliferation will be quantified by colorimetric intensity using a microplate reader (Gemini EM) and compared to a standard curve. In addition, proliferation will be assayed by tritiated thymidine incorporation. At each time point (24, 48 and 72 h), the PEG/PNPIAAm hydrogels will be removed from the cell monolayers and cell proliferation will be measured by incubating the cells in a tritiated thymidine solution. Proliferation will then be determined using a scintillation counter measuring the amount of radioactivity in the wells.

In vivo murine wound healing assays will be assessed. Paired full-thickness skin wounds will be made on the dorsum of 12-week old C57/BL6J (control) or db/db (diabetic) mice. Wounds will be stented with silicone rings having 10 mm outer diameter and 6 mm inner diameter to inhibit healing by contraction. PEG/PNIPAAm hydrogels swollen with a vulnerary agent, or no vulnerary agent will be applied to mice from each group every 24 h. The rate and extent of wound healing will be monitored by digital images taken at 0, 3, 7, 10, 14, and 24 days, or until time of closure, i.e., when the wound bed is substantially filled with new tissue. At each time point, 3 mice from each group will be euthanized and the wound area excised for histological processing. Digital images of the sections will be analyzed for epithelial gap and total area of granulation tissue. The wound areas will be quantified using MetaXpress software. To demonstrate in vivo cell proliferation, a subset of mice will be injected intraperitoneally with 100 mg of bromodeoxyuridine (BrdU) (available from Sigma Aldrich) 3 h before euthanizing. Wounds will be harvested, processed, and proliferating cells detected with immunohistochemistry using a biotyinylated anti-BrdU antibody (available from Roche Applied Science).

### Example 5

Photopatterning will be used to form a device body having an absorptive portion comprising a PEG hydrogel and a delivery portion comprising a PEG/PNIPAAm IPN hydrogel and EGF. A PEG hydrogel having a thickness of about 1000 μm will be formed by photopolymerization and photocrosslinking of PEG-DA as described above for Example 4. The PEG hydrogel will then be swollen with an aqueous solution of a PNIPAAm polymer precursor and photoinitiator as described above for Example 4. The aqueous solution will also contain EGF tethered to a cross-linking agent suitable for use with PEG, such as described herein. The swollen PEG hydrogel will then be irradiated with a UV light source as in Example 4, except that the light will be passed through an opaque grating with spacing of 1 mm inserted between the light source and the swollen PEG hydrogel. The resulting hydrogel will be washed for about 48 hours in deionized water to remove any residual photoinitiator or monomer and dried in a desiccator for at least about 12 hours. The irradiated regions will be cross-linked PEG/PNIPAAm IPN hydrogels incorporating EGF and will comprise a delivery portion of the device body. The non-irradiated regions will be PEG hydrogel and will comprise an absorptive portion of the device body.

This invention is illustrative and not limiting. Further modifications will be apparent to one skilled in the art in light of this disclosure and such modifications are intended to fall within the scope of the appended claims. Each publication and patent application cited in the specification is incorporated herein by reference in its entirety as if each individual publication or patent application were specifically and individually put forth herein.

As one of ordinary skill in the art will appreciate, various changes, substitutions, and alterations could be made or otherwise implemented without departing from the principles of the present invention. Accordingly, the scope of the invention should be determined by the following claims and their legal equivalents.

1. A device for assisting in wound healing, comprising:
an interpenetrating polymer network hydrogel comprising:
a first polymer network comprising a hydrophilic nonionic polymer cross-linked between ends; and
a second polymer network that has been cross-linked and/or polymerized in the presence of the first polymer network,
wherein the hydrogel is configured to absorb wound exudates from a wound and is pre-wetted with a predetermined amount of a wetting agent so as to be nonadhesive to surrounding tissue.

2. The device of claim 1, wherein the wetting agent comprises a saline solution, water or glycerol.

3. The device of claim 1, wherein the hydrogel comprises at least about 10% by weight of the wetting agent.

4. The device of claim 1, wherein the first polymer network comprises poly(ethylene glycol).

5. The device of claim 4, wherein a segment of the poly(ethylene glycol) between end-links has a molecular weight in the range of about 1 kDa to about 20 kDa.

6. The device of claim 4, wherein a segment of the poly(ethylene glycol) between end-links has a molecular weight in the range of about 3 kDa to about 8 kDa.

7. The device of claim 1, wherein the second polymer network comprises poly(acrylic acid).

8. The device of claim 1, wherein the hydrogel is configured to deliver one or more vulnerary agents to the wound, wherein the one or more vulnerary agents is selected from the group consisting of growth factors, antifungals, antibiotics, antivirals, anti-parasitic agents, vitamins, pH buffering agents or compositions, enzymes, antibodies, and combinations thereof, and wherein the delivery to the wound is in response to an environmental stimulus, wherein the environmental stimulus is selected from the group consisting of temperature, pH, pK, electromagnetic radiation, electric field, ultrasound energy, and combinations thereof.

9. The device of claim 8, wherein the vulnerary agent comprises an engineered growth factor.

10. The device of claim 8, wherein the growth factor is selected from the group consisting of: a platelet-derived growth factor (PDGF), an epidermal growth factor (EGF), a transforming growth factor (TGF), a basic fibroblast growth factor (FGF1), an acidic fibroblast growth factor (FGF2), a bone morphogenetic protein (BMP), hydroxylapatite, a hepatocyte growth factor (HGF), a vascular endothelial growth factor (VEGF), a granulocyte-colony stimulating factor (G-CSF), a granulocyte macrophage colony stimulating factor (GM-CSF), a neurotrophin, erythropoietin (EPO), thrombopoietin (TPO), and combinations thereof.

11. The device of claim 10, wherein the neurotrophin is nerve growth factor (NGF) or neurotrophin-3 (NT-3).

12. The device of claim 10, wherein the transforming growth factor is growth differentiation factor-8 (GDF-8) or growth differentiation factor-9 (GDF-9).

13. The device of claim 1, configured to be removably secured to skin proximate the wound.

14-42. (canceled)
